(19) United States
(12) Patent Application Publication
(10) Pub. No.: US 2015/0033414 A1
(43) Pub. Date: Jan. 29, 2015

(54) FLOWERING MODIFICATION IN JATROPHA AND OTHER PLANTS

(71) Applicant: TEMASEK LIFE SCIENCES LABORATORY LIMITED, Singapore (SG)

(72) Inventors: Nam-Hai Chua, Singapore (SG); Jian Ye, Singapore (SG); Yun-Feng Geng, Singapore (SG); Bipei Zhang, Singapore (SG)

(73) Assignee: TEMASEK LIFE SCIENCES LABORATORY LIMITED, Singapore (SG)

(21) Appl. No.: 14/381,311

(22) PCT Filed: Feb. 27, 2013
(86) PCT No.: PCT/SG2013/000078

§ 371 Date: Aug. 27, 2014

Related U.S. Application Data
(60) Provisional application No. 61/603,458, filed on Feb. 27, 2012.

(51) Int. Cl. C12N 15/82; C07K 14/415

(52) U.S. Cl. CPC C12N 15/827; C07K 14/415; C12N 15/8202
USPC 800/290; 536/23.6; 530/370; 435/320.1; 435/419; 800/298; 800/278

(57) ABSTRACT

The present invention relates to the field of modifying Jatropha branching, flowering time, fruiting and seed setting. More specifically, the present invention relates to a method for control and optimization of Jatropha curcas flowering time, fruiting and seed production by manipulating one gene from Jatropha. The present invention can be used in various Jatropha breeding efforts.

### SEQUENCE SUBMISSION

The present application is being filed along with a Sequence Listing in electronic format. The Sequence Listing is entitled 2577213PCTSequenceListing.txt, created on 25 Feb. 2013 and is 86 kb in size. The information in the electronic format of the Sequence Listing is incorporated herein by reference in its entirety.

### BACKGROUND OF THE INVENTION

The present invention relates to the field of modifying Jatropha branching, flowering time, fruiting and seed setting. More specifically, the present invention relates to a method for control and optimization of Jatropha curcas flowering time, fruiting and seed production by manipulating one gene from Jatropha. The present invention can be used in various Jatropha breeding efforts.

The publications and other materials used herein to illuminate the background of the invention or provide additional details respecting the practice, are incorporated by reference, and for convenience are respectively grouped in the Bibliography.

The world is confronted with a dwindling supply of fossil fuel and worsening Green House Effect. There is an urgent demand to increase the production and consumption of renewable energy. Biofuels have been recognized as a national priority for many countries in their search for alternative sources to meet their energy security needs and at the same time help reduce CO₂ emissions that cause the Green House Effect. The demand for biofuel has put increasing pressure on food production. For example, to satisfy the biofuel need for Germany in 2017 as mandated by the German government the entire farm land of this country would have to be used for growing bioenergy crops with no land left for food production. To ease this competition between food and fuel for land and to satisfy our need for renewable fuels, it is necessary to use marginal land for bio-energy production.

Jatropha curcas is a small woody plant belonging to the Euphorbiaceae family. Several unique characteristics of Jatropha curcas make it an ideal plant for biodiesel production. These include its rapid growth, ease of propagation, low seed cost, high oil content, short gestation period, wide adaptability, drought tolerance and the ability to thrive on degraded soils. Moreover, the relatively short stature of Jatropha plants renders convenient collection of seed (Jones, 1991; Sujatha et al., 2008).

At a certain point in their life cycle, annual plants undergo a major developmental transition and switch from vegetative to reproductive development. As this process is rarely reversible ensuring that the timing of this transition is optimal for pollination and seed development is a major factor in reproductive success. Physiological and genetic analysis of flowering has shown that multiple environmental and endogenous inputs influence the timing of the switch from vegetative to reproductive development. The molecular identity of these different inputs is being dissected using molecular genetic approaches in the model plant Arabidopsis (Srikanth and Schmid 2011). Changes of length of light/dark period, temperature, light quality and light fluences will affect plant flowering process. There are multiple pathways that quantitatively regulate an overlapping set of common targets, the floral pathway integrators, whose activities convert the shoot apical meristem to a reproductive fate. An emerging theme is that changing the predominance of these input pathways could account for much of the plasticity and diversity of flowering time control within and between plant species (Blackman et al. 2010; Pin et al. 2010).

Flowering time and the female to male flower ratio in Jatropha determine the final yield of this crop. Jatropha is monoecious—with male and female flowers on the same plant and borne by the same inflorescence. Although the number of male and female flowers per inflorescence varies widely among different experiments (male: 25-238 and female: 1-19), the male-to-female flower ratio is similar between the two reported studies (25:1-29:1) (Achten et al. 2010).

Little research has been done on the genetic mechanism regulating flowering time or controlling the female/male flower ratio in Jatropha (Achten et al. 2010). To precisely control the time of flowering, plants have evolved mechanisms to integrate seasonally predictable environmental cues (such as changes in photoperiod and prolonged periods of cold temperatures) and developmental cues (such as maturity). To allow this diversity of environmental cues to influence when flowering occurs in Arabidopsis, multiple pathways converge on a small number of genes, the floral integrator genes, including the floral promoters FLOWERING LOCUS T (FT) and TWIN SISTER OF FT (TSF) (Imaizumi 2010). FT and TSF are members of a family of proteins that contain a plant specific phosphatidylethanolamine-binding protein (PEBP) domain. In Arabidopsis, the PEBP family is divided intro three subfamilies, FT-like, TERMINAL FLOWER1 (TFL1)-like, and Mother of FT and TFL1 (MFT)-like. FT and TFL1 are thought to be molecular switches for vegetative growth to reproductive development and MFT is phylogenetically ancestral to them (Kobayashi and Weigel 2007). In other plants, e.g. tomato, the balance between the activity of the tomato FT homolog SINGLE FLOWER TRUSS and that of the TFL1 homolog SELF-FRUNING affects a variety of development processes, such as flowering response, reiterative growth, termination cycles, leaf maturation, and stem growth (Carmel-Goren et al. 2003; Lifschitz et al. 2006). In sugarbeet, flowering time is found to be controlled by the interplay of two paralogs of the FLOWERING LOCUS T (FT) gene in Arabidopsis that have evolved antagonistic functions. BvFT2 is functionally conserved with FT and essential for flowering. By contrast, BvFT1 functions as a flowering terminator (such as AtTFLJ) to represses flowering and its down-regulation is crucial for the vernalization response in beets. Therefore, all three subfamilies of PEBP genes can function as general developmental regulators rather than simple floral initiators or florigens (Pin et al. 2010).

No flowering genes have yet been isolated from industrial plants, e.g. key industrial plants from the Euphorbiaceae, including prominent plants include Cassava (Manihot esculenta), Castor oil plant (Ricinus communis), tung tree (Vernicia fordii), physics nut (Jatropha curcas), and the rubber tree (Hevea brasiliensis). Many are grown as ornamental plants, such as Poinsettia (Euphorbia pulcherrima). Leafy spurge (Euphorbia esula) and Chinese tallow (Sapium sebiferum) are invasive weeds in North America. In medicine, some species of Euphorbiaceae proved to be effective against genital herpes (HSV-2). Furthermore, the difficulties of transforming Euphorbiaceae hinder their manipulation by genetic modification and improvements. Therefore, there has been no successful report on the control of flowering time of industrial plants using genes that promote flowering. It should be noted that given their long life cycle it is very time consuming to use traditional breeding methods to breed plants with optimal flowering times and male/female flower ratio.

Thus, it is desired to be able to control the flowering time and male/female flower ratio of plants with significant industrial importance; in particular, trees and shrubs in the Euphorbiaceae family, such as Cassava (Manihot esculenta), Castor oil plant (Ricinus communis), tung tree (Vernicia fordii), physics nut (Jatropha curcas), and the rubber tree (Hevea brasiliensis). Many are grown as ornamental plants, such as Poinsettia (Euphorbia pulcherrima). Others such as leafy spurge (Euphorbia esula) and Chinese tallow (Sapium sebiferum) are invasive weeds in North America.

### SUMMARY OF THE INVENTION

The present invention relates to the field of modifying Jatropha branching, flowering time, fruiting and seed setting. More specifically, the present invention relates to a method for control and optimization of Jatropha curcas flowering time, fruiting and seed production by manipulating one gene from Jatropha. The present invention can be used in various Jatropha breeding efforts.

In a first aspect, the present invention provides an isolated nucleic acid encoding a JcFT protein comprising the amino acid sequence set forth in SEQ ID NO:2. In one embodiment, the nucleic acid comprises the nucleotide sequence set forth in SEQ ID NO:1. In another embodiment, the nucleic acid comprises the nucleotide sequence set forth in SEQ ID NO:3. In a further embodiment, the nucleic acid encodes a variant JcFT protein. In one embodiment, the variant JcFT protein has at least 90% sequence identity with the JcFT protein while having the activity of the JcFT protein. In another embodiment, the variant JcFT protein has one or more amino acid changes in the amino acid sequence of the JcFT protein while having the activity of the JcFT protein. In one embodiment, the nucleic acid further comprises a plant operable promoter operably linked to the coding sequence.

In a second aspect, the present invention provides an isolated nucleic acid encoding a JcTFL1L-1 protein comprising the amino acid sequence set forth in SEQ ID NO:33. In one embodiment, the nucleic acid comprises the nucleotide sequence set forth in SEQ ID NO:32. In another embodiment, the nucleic acid comprises the nucleotide sequence set forth in SEQ ID NO:34. In a further embodiment, the nucleic acid encodes a variant JcTFL1L-1 protein. In one embodiment, the variant JcTFL1L-1 protein has at least 90% sequence identity with the JcTFL1L-1 protein while having the activity of the JcTFL1L-1 protein. In another embodiment, the variant JcTFL1L-1 protein has one or more amino acid changes in the amino acid sequence of the JcTFL1L-1 protein while having the activity of the JcTFL1L-1 protein. In one embodiment, the nucleic acid further comprises a plant operable promoter operably linked to the coding sequence.

In a third aspect, the present invention provides an isolated nucleic acid encoding a JcTFL1L-2 protein comprising the amino acid sequence set forth in SEQ ID NO:36. In one embodiment, the nucleic acid comprises the nucleotide sequence set forth in SEQ ID NO:35. In another embodiment, the nucleic acid comprises the nucleotide sequence set forth in SEQ ID NO:37. In a further embodiment, the nucleic acid encodes a variant JcTFL1L-2 protein. In one embodiment, the variant JcTFL1L-2 protein has at least 90% sequence identity with the JcTFL1L-2 protein while having the activity of the JcTFL1L-2 protein. In another embodiment, the variant JcTFL1L-2 protein has one or more amino acid changes in the amino acid sequence of the JcTFL1L-2 protein while having the activity of the JcTFL1L-2 protein. In one embodiment, the nucleic acid further comprises a plant operable promoter operably linked to the coding sequence.

In a fourth aspect, the present invention provides a construct or vector comprising an isolated nucleic acid as described herein. In one embodiment, the construct or vector is an expression construct or vector. In another embodiment, the construct or vector further comprises a selectable marker. In a further embodiment, the construct or vector comprises a Cre-lox recombination marker free system.

In a fifth aspect, the present invention provides a transgenic plant comprising a nucleic acid, construct or vector described herein. In one embodiment, the transgenic plant may be any plant species. In another embodiment, the transgenic plant may be a plant of the Euphorbiaceae family. In a further embodiment, the transgenic plant may be a Jatropha plant.

In a sixth aspect, the present invention provides a method of controlling, regulating or altering the flowering time in a plant using a nucleic acid, construct or vector described herein. In one embodiment, the transgenic plant may be any plant species. In another embodiment, the transgenic plant may be a plant of the Euphorbiaceae family. In a further embodiment, the transgenic plant may be a Cassava (Manihot esculenta), a Castor oil plant (Ricinus communis), a tung tree (Vernicia fordii), a physics nut (Jatropha curcas), or a rubber tree (Hevea brasiliensis).

### BRIEF DESCRIPTION OF THE FIGURES

FIGS. 1A and 1B show nucleotide sequence (SEQ ID NO:1) and encoded amino acid sequence (SEQ ID NO:2) of FLOWERING LOCUS T (FT) in Jatropha and genomic organization of the JcFT. Boxes and lines represent exonic and intronic regions, respectively. Box and line individually indicate exon and intron regions. Numbers showed the base pairs of nucleotides. Red box indicates 3′ or 5′ untranslated regions associated with the expression of the gene product.

FIGS. 2A and 2B show CaMV 35 promoter driven (FIG. 2A) and β-estradiol mediated Cre-lox marker free vector (FIG. 2B) for Jatropha transformation.

FIGS. 3A-3E show various transgenic Arabidopsis plants expressing JcFT with early-flowering phenotypes. FIG. 3A: Left pot: ft-10; right pot: 35S:JcFT/ft-10. FIG. 3B: Col-0 WT control. FIG. 3C-3E: 35S:JcFT/Col-0 WT lines. Bars: 10 mm.

FIG. 4 shows RT-PCR analysis of JcFT and AtFT transcripts from transgenic Arabidopsis plants. Total RNAs were extracted from leaves of 25 day-old plants.

FIG. 5 shows overexpression of JcFT strongly accelerates flowering in transgenic Jatropha plants

FIGS. 6A-6F show early flowering phenotypes in JcFT-overexpressing transgenic Jatropha. FIG. 6A: Six-month old WT Jatropha plant. FIGS. 6B-6F: Three-month old JcFT transgenic plants after transplanting into soil. Bar: 10 cm.

FIG. 7 shows days after soiling for JcFT-overexpressing transgenic Jatropha and WT control plants.

FIGS. 8A-8E shows more branches and fruits in JcFT-overexpressing transgenic Jatropha. FIG. 8A: WT control. FIGS. 8B and 8C: JcFT overexpressing Jatropha lines showing more branches and fruits. FIG. 8D shows similar fruit size between transgenic plants and WT control. Bar: FIGS. 8A, 8B and 8C—10 cm; FIG. 8D—10 mm. FIG. 8E summarizes the data.

FIG. 9 shows the RT-PCR analysis of JcFT-overexpressing transcripts in transgenic Jatropha lines.

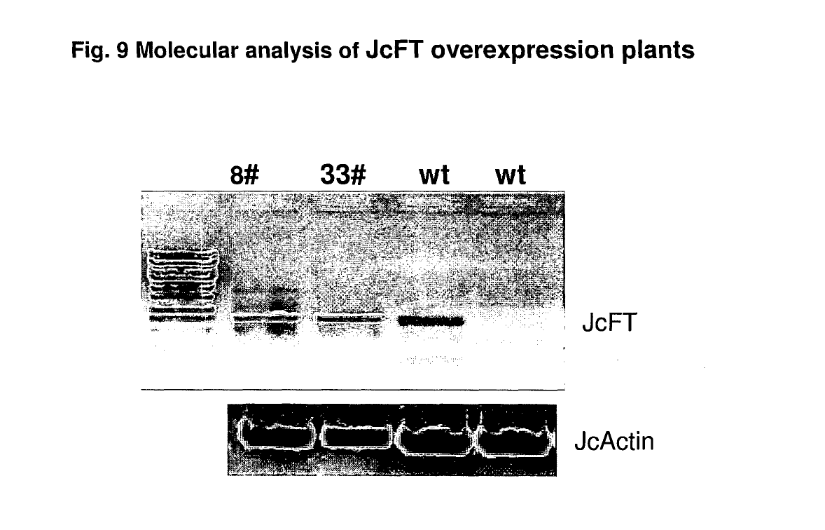

FIG. 10 shows a schematic description of transformation vector and probe localization

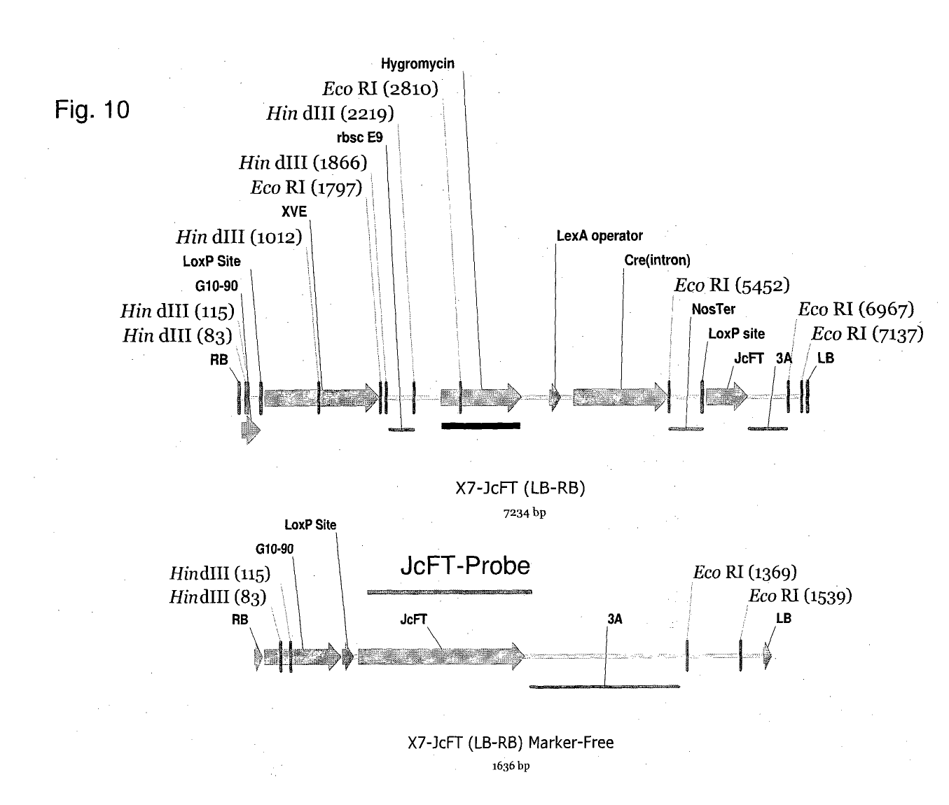

FIG. 11 shows Southern blotting analysis of trangenic Jatropha lines with JcFT probe (left) or hygromycin phosphotransferase (hgy) probe (right).

FIG. 12 shows heritable Early flowering phenotypes in T1 plants of JcFT overexpressing Jatropha line.

FIG. 13 shows the identification of Euphorbianceae PEBP homologs. Amino acid sequence alignment of PEBP family members. Red stars on the upper row indicate the Tyr85(Y)/His88(H) and Gln140(Q)/Asp144(D) residues distinguishing all FT-like and TFL1/CEN-like members. The amino acid sequences for the family members from top to bottom are SEQ ID NO:5 to SEQ ID NO:19, respectively.

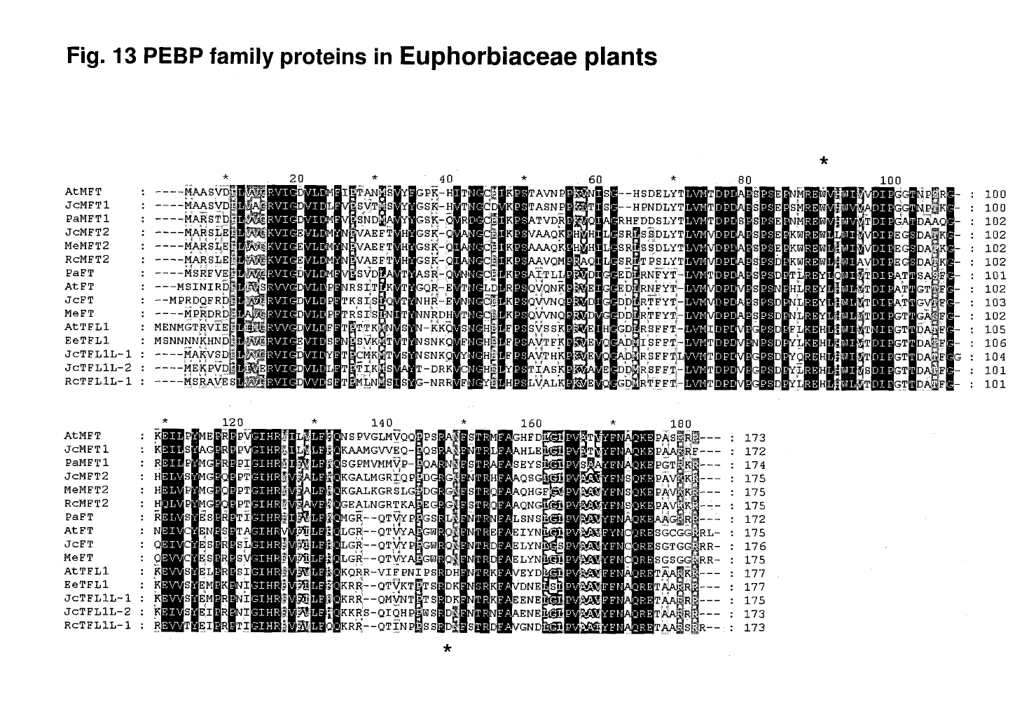

FIG. 14 shows a phylogenetic analysis of PEBP family proteins in Euphorbiaceae plants. A Neighbor Joining phylogenetic analysis of multiple members of the PEBP family from several plant species including the sugar beet homologs. Bootstrap values for 1000 re-samplings are shown on each branch.

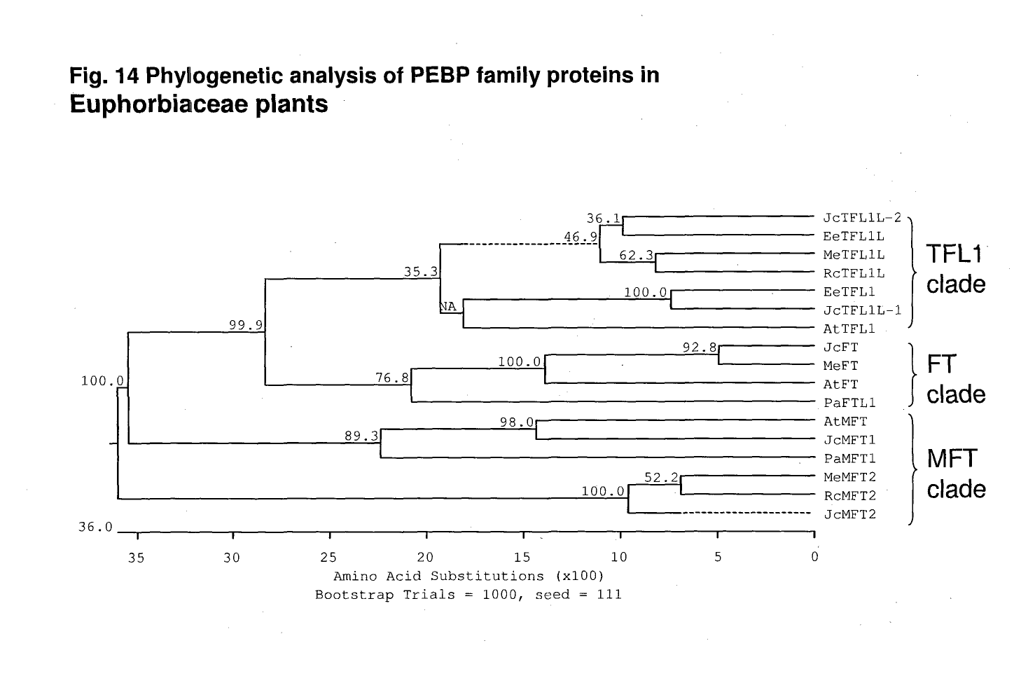

FIG. 15 shows early instead of late flowering phenotypes of transgenic Arabidopsis plants overexpressing JcTFL1L-1 and JcTFL1L-2.

FIGS. 16A and 16B show RT-PCR analysis of JcTFL1L-1:3 HA (FIG. 16A) and JcTFL1L-2:3 HA (FIG. 16B) transcripts in transgenic Arabidopsis

FIGS. 17A and 17B show Western blot analysis of JcTFL1L-1:3 HA (FIG. 17A) and JcTFL1L-2:3 HA (FIG. 17B) protein translation

FIGS. 18A, 18B and 18C show early flowering and self-pruning phenotypes in JcTFL1L-1 overexpression Jatropha

FIG. 19 shows multiple shoots phenotype in JcTFL1L-2 overexpression Jatropha

### DETAILED DESCRIPTION OF THE INVENTION

The present invention relates to the field of modifying Jatropha branching, flowering time, fruiting and seed setting. More specifically, the present invention relates to a method for control and optimization of Jatropha curcas flowering time, fruiting and seed production by manipulating one gene from Jatropha. The present invention can be used in various Jatropha breeding efforts.

Unless defined otherwise, all technical and scientific terms used herein have the same meaning as is commonly understood by one of skill in the art to which the invention belongs.

As used herein, “allele” refers to any of one or more alternative forms of a gene locus, all of which alleles relate to a trait or characteristic. In a diploid cell or organism, the two alleles of a given gene occupy corresponding loci on a pair of homologous chromosomes.

As used herein, “JcFT” refers to the Jatropha curcus Flowering locus T gene or its encoded protein. JcFT protein may also be referred to as Jatropha curcas FT protein”.

As used herein, “JcTFL1L-1” refers to the Jatropha curcus Terminal Flower1 locus 1 gene or its encoded protein. JcTFL1L-1 protein may also be referred to as Jatropha curcas TFL1L-1 protein”.

As used herein, “JcTFL1L-2” refers to the Jatropha curcus Terminal Flower1 locus 2 gene or its encoded protein. JcTFL1L-2 protein may also be referred to as Jatropha curcas TFL1L-2 protein”.

As used herein, “gene” refers to a nucleic acid sequence that encompasses a 5′ promoter region associated with the expression of the gene product, any intron and exon regions and 3′ or 5′ untranslated regions associated with the expression of the gene product.

As used herein, “genotype” refers to the genetic constitution of a cell or organism.

As used herein, “phenotype” refers to the detectable characteristics of a cell or organism, which characteristics are the manifestation of gene expression.

The terms “polynucleotide,” nucleic acid” and “nucleic acid molecule are used in therchangeably herein to refer to a polymer of nucleotides which may be a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. Amino acids may be referred to by their commonly known three-letter or one-letter symbols. Amino acid sequences are written left to right in amino to carboxy orientation, respectively. Numeric ranges are inclusive of the numbers defining the range.

So far, there has been is no report about a TFL1 functioning as a flowering promoter or florigen. The present invention discovered genes of the Jatropha PEBP family and their use in the control of flowering time, fruiting, branching, and female/male flower ratio in the model plant Arabidopsis and Jatropha. Interestingly, two Jatropha TFL1 proteins were discovered that actually function as flowering promoters or florigens rather than flowering repressors.

For model organisms such as A. thaliana, much work has been done to identify and characterize genes that flowering time. However, little has been done on similar genes in industrial trees. Moreover, the homology of gene sequences between the mustard family (Brassicaceae), of which A. thaliana is a member, and Euphorbiaceae is low because of the wide evolutionary distance between the two families. It is also not clear whether genes of limited sequence similarity may have similar biological function. Thus, the present invention isolated genes that encode proteins with a phosphatidylethanolamine-binding) domain and tested their effects on flowering time control in Arabidopsis and Jatropha. Specifically, key flowering control genes from Jatropha, such as FT, Terminal of Flowering1 (TFL1) and Mother of FT (MFT), all members of a family of proteins that contain a phosphatidylethanolamine-binding protein (PEBP) domain, are the subject of the present invention.

Thus, in a first aspect, the present invention provides an isolated nucleic acid encoding a JcFT protein comprising the amino acid sequence set forth in SEQ ID NO:2. In one embodiment, the nucleic acid comprises the nucleotide sequence set forth in SEQ ID NO:1. In another embodiment, the nucleic acid comprises the nucleotide sequence set forth in SEQ ID NO:3. In a further embodiment, the nucleic acid encodes a variant JcFT protein. In one embodiment, the variant JcFT protein has at least 90%, preferably at least 95%, more preferably at least 98%, most preferably 99%, sequence identity with the JcFT protein while having the activity of the JcFT protein. In another embodiment, the variant JcFT protein has one or more amino acid changes in the amino acid sequence of the JcFT protein while having the activity of the JcFT protein. In one embodiment, the nucleic acid further comprises a plant operable promoter operably linked to the coding sequence.

In a second aspect, the present invention provides an isolated nucleic acid encoding a JcTFL1L-1 protein comprising the amino acid sequence set forth in SEQ ID NO:33. In one embodiment, the nucleic acid comprises the nucleotide sequence set forth in SEQ ID NO:32. In another embodiment, the nucleic acid comprises the nucleotide sequence set forth in SEQ ID NO:34. In a further embodiment, the nucleic acid encodes a variant JcTFL1L-1 protein. In one embodiment, the variant JcTFL1L-1 protein has at least 90%, preferably at least 95%, more preferably at least 98%, most preferably 99%, sequence identity with the JcTFL1L-1 protein while having the activity of the JcTFL1L-1 protein. In another embodiment, the variant JcTFL1L-1 protein has one or more amino acid changes in the amino acid sequence of the JcTFL1L-1 protein while having the activity of the JcTFL1L-1 protein. In one embodiment, the nucleic acid further comprises a plant operable promoter operably linked to the coding sequence.

In a third aspect, the present invention provides an isolated nucleic acid encoding a JcTFL1L-2 protein comprising the amino acid sequence set forth in SEQ ID NO:36. In one embodiment, the nucleic acid comprises the nucleotide sequence set forth in SEQ ID NO:35. In another embodiment, the nucleic acid comprises the nucleotide sequence set forth in SEQ ID NO:37. In a further embodiment, the nucleic acid encodes a variant JcTFL1L-2 protein. In one embodiment, the variant JcTFL1L-2 protein has at least 90%, preferably at least 95%, more preferably at least 98%, most preferably 99%, sequence identity with the JcTFL1L-2 protein while having the activity of the JcTFL1L-2 protein. In another embodiment, the variant JcTFL1L-2 protein has one or more amino acid changes in the amino acid sequence of the JcTFL1L-2 protein while having the activity of the JcTFL1L-2 protein. In one embodiment, the nucleic acid further comprises a plant operable promoter operably linked to the coding sequence.

In some embodiments, the polynucleotide may be one encoding a polypeptide of a variant of the amino acid sequences disclosed herein, which variant is an amino acid sequence disclosed herein having one or several amino acid residues substituted, deleted, inserted and/or added. The site at which one or several amino acid residues are substituted, deleted, inserted and/or added may be any site in the amino acid sequence, as long as the polypeptide with one or several amino acid residues substituted, deleted, inserted and/or added has the function of regulating the flowering time of a plant. As used herein, the term “one or several amino acid residues” refers specifically to up to 10 amino acid residues in number, preferably to up to 6 amino acid residues, more preferably to up to 2 amino acid residues and even more preferably to one amino acid residue.

When the amino acids are altered, for example, by substitution, it is preferable to be conservatively substituted. This means that a particular amino acid residue is substituted with a different amino acid in which the properties of the amino acid side-chain are conserved. Non-limited examples of such the conservative substitution include substitution between hydrophobic amino acids such as alanine, isoleucine, leucine, methionine, phenylalanine, proline, tryptophan, tyrosine and valine, substitution between hydrophilic amino acids such as arginine, aspartic acid, asparagine, cysteine, glutamic acid, glutamine, glycine, histidine, lysine, serine and threonine, substitution between amino acids having an aliphatic side chain such as glycine, alanine, valine, leucine, isoleucine and proline, substitution between amino acids having a hydroxy-containing side chain such as serine, threonine and tyrosine, substitution between amino acids having a sulfur atom-containing side chain such as cysteine and methionine, substitution between amino acids having a carboxylic acid- and amide-containing side chain such as aspartic acid, asparagine, glutamic acid and glutamine, substitution between amino acids having a base-containing side chain such as arginine, lysine and histidine, and substitution between amino acids having an aromatic-containing side chain such as histidine, phenylalanine, tyrosine and tryptophan. The substitutions between amino acids having the same amino acid side-chain properties may retain the biological activity of the polypeptide.

In some embodiments, the polynucleotide may be a variant of a polynucleotide selected from the group consisting of the polynucleotides described herein, which variant has one to 30 nucleotides substituted, deleted, inserted and/or added. The site at which nucleotides are substituted, deleted, inserted and/or added may be any site, as long as the polynucleotide with substituted, deleted, inserted and/or added nucleotides has the function of regulating the flowering time of a plant.

Examples of methods for preparing a nucleic acid encoding a protein comprising altered amino acids are well known to those skilled in the art, including site-directed mutagenesis (Kramer and Fritz, 1987). Examples of specific methods for altering nucleotides also include methods using a commercially available kit (e.g. Transformer Site-Directed Mutagenesis Kit: Clonetech; QuickChange Site Directed Mutagenesis Kit: Stratagene) and methods using polymerase chain reaction (PCR). These methods are well known to those skilled in the art. The amino acid sequence of a protein may also be mutated in nature due to the mutation of a nucleotide sequence. A nucleic acid encoding proteins having the amino acid sequence of a natural JcFT, JcTFL1L-1 or JcTFL1L-2 proteins wherein one or more amino acids are substituted, deleted, and/or added are also included in the polynucleotide of the present invention, so long as they encode a protein functionally equivalent to a natural JcFT, JcTFL1L-1 or JcTF11L-2 protein. Also natural JcFT, JcTFL1L-1 or JcTF11L-2 protein homologs in related Euphorbiaceae plants which show high identities to the sequences of the JcFT, JcTFL1L-1 or JcTF11L-2 proteins are also included in the polynucleotide of the present invention, so long as they encode a protein functionally equivalent to a natural JcFT, JcTFL1L-1 or JcTF11L-2 protein. Additionally, nucleotide sequence variants that do not give rise to amino acid sequence changes in the protein (degeneracy variants) are also included in the polynucleotide of the present invention.

In a fourth aspect, the present invention provides a construct or vector comprising an isolated nucleic acid as described herein. In one embodiment, the construct or vector is an expression construct or vector. In another embodiment, the construct or vector further comprises a selectable marker. In a further embodiment, the construct or vector comprises a Cre-lox recombination marker free system, such as well known in the art.

The construct typically includes regulatory regions operatively linked to the 5′ side of the nucleic acid described herein (such as a nucleic acid encoding a JcTFL1L-1 protein) and/or to the 3′ side of the nucleic acid. A cassette containing all of these elements is also referred to herein as an expression cassette. The expression cassettes may additionally contain 5′ leader sequences in the expression cassette construct. The regulatory regions (i.e., promoters, transcriptional regulatory regions, and translational termination regions) and/or the polynucleotide encoding a signal anchor may be native/analogous to the host cell or to each other. The promoters and tissue-specific promoters are particularly useful for preparing constructions for the transformation of Jatropha, as well as for the transformation of other oil crops. Alternatively, the regulatory regions and/or the polynucleotide encoding a signal anchor may be heterologous to the host cell or to each other. See, U.S. Pat. No. 7,205,453 and U.S. Patent Application Publication Nos. 2006/0218670, 2006/0248616 and 20090100536, and the references cited therein. The expression cassettes may additionally contain 5′ leader sequences in the expression cassette construct. Such leader sequences can act to enhance translation. Translation leaders are known in the art and include those described in International Publication No. WO 2008/094127 and the references cited therein.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (WO 99/48338 and U.S. Pat. No. 6,072,050); the core CaMV 35S promoter (Odell et al., 1985); rice actin (McElroy et al., 1990); ubiquitin (Christensen and Quail, 1989; Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; and 5,608,142.

Other promoters include inducible promoters, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. Other promoters include those that are expressed locally at or near the site of pathogen infection. In further embodiments, the promoter may be, a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters are described in U.S. Pat. Nos. 6,506,962, 6,575,814, 6,972,349 and 7,301,069 and in U.S. Patent Application Publication Nos. 2007/0061917 and 2007/0143880.

Generally, the expression cassette may additionally comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or basta (e.g., the bar gene). See generally, International Publication No. WO 02/36782, U.S. Pat. No. 7,205,453 and U.S. Patent Application Publication Nos. 2006/0218670, 2006/0248616, 2007/0143880 and 2009/0100536, and the references cited therein. See also, Jefferson et al. (1991); De Wet et al. (1987); Goff et al. (1990); Kain et al. (1995) and Chiu et al. (1996). This list of selectable marker genes is not meant to be limiting. Any selectable marker gene can be used. The selectable marker gene is also under control of a promoter operable in the plant species to be transformed. Such promoters include those described in International Publication No. WO 2008/094127 and the references cited therein.

Alternatively, the expression cassette may additionally comprise a Cre-lox recombination marker free system, such as described herein. Such a system is useful for producing selection marker free transgenic Jatropha plants or other plants.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

In a fifth aspect, the present invention provides a transgenic plant comprising a nucleic acid, construct or vector described herein. In one embodiment, the transgenic plant may be any plant species. In another embodiment, the transgenic plant may be a plant of the Euphorbiaceae family. In a further embodiment, the transgenic plant may be a Cassava (Manihot esculenta), a Castor oil plant (Ricinus communis), a tung tree (Vernicia fordii), a physics nut (Jatropha curcas), or a rubber tree (Hevea brasiliensis).

Once a nucleic acid has been cloned into an expression vector, it may be introduced into a plant cell using conventional transformation procedures. The term “plant cell” is intended to encompass any cell derived from a plant including undifferentiated tissues such as callus and suspension cultures, as well as plant seeds, pollen or plant embryos. Plant tissues suitable for transformation include leaf tissues, root tissues, meristems, protoplasts, hypocotyls, cotyledons, scutellum, shoot apex, root, immature embryo, pollen, and anther. “Transformation” means the directed modification of the genome of a cell by the external application of recombinant DNA from another cell of different genotype, leading to its uptake and integration into the subject cell's genome. In this manner, genetically modified plants, plant cells, plant tissue, seed, and the like can be obtained.

DNA constructs containing the nucleic acids of the present invention can be used to transform any plant and particularly Jatropha plants or plants of the Euphorbiaceae. The constructs may be introduced into the genome of the desired plant host by a variety of conventional techniques. Techniques for transforming a wide variety of higher plant species are well known and described in the technical and scientific literature. Transformation protocols may vary depending on the type of plant or plant cell, i.e., monocot or dicot, targeted for transformation, as is well known to the skilled artisan. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Thus, any method, which provides for effective transformation/transfection may be employed. See, for example, U.S. Pat. Nos. 7,241,937, 7,273,966 and 7,291,765 and U.S. Patent Application Publication Nos. 2007/0231905 and 2008/0010704 and references cited therein. See also, International Published Application Nos. WO 2005/103271 and WO 2008/094127 and references cited therein. Techniques which have been used to transform oil palm include biolistic-mediated transformation and Agrobacterium-mediated transformation. See, for example, Masli et al. (2009); Omidvar et al. (2008); Parveez et al. (2008); Abdullah et al. (2005); Parveez et al. (2000); Chowdhury, et al. (1997); and U.S. Patent Application Publication No. 2009/0038032. In addition, transformation of Jatropha has been described in International Publication No. 2010/071608.

Transformed plant cells which are derived by any of the above transformation techniques can be cultured to regenerate, a whole plant which possesses the transformed genotype and thus the desired phenotype, e.g., a transgenic plant. A “transgenic plant” is a plant into which foreign DNA has been introduced. A “transgenic plant” encompasses all descendants, hybrids, and crosses thereof, whether reproduced sexually or asexually, and which continue to harbor the foreign DNA. Regeneration techniques rely on manipulation of certain phytohormones in a tissue culture growth medium, typically relying on a biocide and/or herbicide marker which has been introduced together with the desired nucleotide sequences. See for example, International Published Application No. WO 2008/094127 and references cited therein.

The foregoing methods for transformation are typically used for producing a transgenic variety in which the expression cassette is stably incorporated. After the expression cassette is stably incorporated in transgenic plants, it can be transferred to other plants by sexual crossing. In one embodiment, the transgenic variety could then be crossed, with another (non-transformed or transformed) variety, in order to produce a new transgenic variety. Alternatively, a genetic trait which has been engineered into a particular cotton line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Any of a number of standard breeding techniques can be used, depending upon the species to be crossed.

Once transgenic plants of this type are produced, the plants themselves can be cultivated in accordance with conventional procedures. Transgenic seeds can, of course, be recovered from the transgenic plants. These seeds can then be planted in the soil and cultivated using conventional procedures to produce transgenic plants. The cultivated transgenic plants will express the DNA of interest in a tissue-preferred or tissue-specific manner as described herein.

In a sixth aspect, the present invention provides a method of controlling, regulating or altering the flowering time in a plant using a nucleic acid, construct or vector described herein. In one embodiment, the transgenic plant may be any plant species. In another embodiment, the transgenic plant may be a plant of the Euphorbiaceae family. In an additional embodiment, the transgenic plant may be a Cassava (Manihot esculenta), a Castor oil plant (Ricinus communis), a tung tree (Vernicia fordii), a physics nut (Jatropha curcas), or a rubber tree (Hevea brasiliensis). In a further embodiment, the transgenic plant may be a Jatropha plant. In some embodiments, the method for controlling the flowering time of plants includes the steps of introducing the polynucleotide, recombinant vector or polypeptide of the present invention into a plant.

In one embodiment, an expression vector described herein is introduced into plant cells to obtain a transgenic plant, in which the flowering is promoted. Even a transgenic plant is not necessary to be obtained. Depending on the host plant, the nucleic acids of the present invention may be introduced into plant cells such that the nucleic acid encoding a flowering protein described herein can be expressed in the plant cells.

In another embodiment, a nucleotide sequence is integrated into the genomic DNA of a plant to enhance the expression of the endogenous flowereing gene. Preferably, examples of such a nucleotide sequence include an expression control sequence. More specifically, examples of such an expression control sequence include promoter sequences and enhancer sequences. Such an expression control sequence is operably integrated into the genomic DNA of a plant to enhance the expression of the endogenous flowering gene in the plant, thereby promoting flowering of the plant.

In a further embodiment, the protein encoded by the flowering gene is introduced into plant cells by injecting the protein into, for example, phloem sap. The protein encoded by the flowering gene migrates to shoot apices and axillary buds to promote flowering of the plant. The protein encoded by the flowering gene may be produced using a recombinant microorganism or extracted from a wild-type plant or transgenic plant. Alternatively, grafting may be performed using, as a rootstock, a plant (for example, a transgenic plant) accumulating the protein encoded by the flowering gene.

In another embodiment, in order to control the flowering time of Jatropha curcas, it is preferable that any of various inducible promoters, for example, a copper ion inducible promoter (e.g. see WO 08/111,661) is ligated to a polynucleotide of the present invention and then introduced into a plant of the order Euphorbiaceae family, in particular, into Jatropha curcas, and that the flowering time of the plant is controlled by exposing the transgenic plant to copper ions. According to this construction, the flowering time of plants can be controlled more effectively as compared with the construction in which, for example, a DNA fragment of the A. thaliana FT gene, which has an amino acid sequence homology of less that 90% with the JcFT protein, is ligated to the above promoter and introduced into a plant. Further, this construct may prevent emergence of undesirable phenotypes, such as morphological aberration.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Materials and Methods

Explant Material for Transformation:

Seeds were obtained from Jatropha curcas (Jc-MD) elite plants pre-selected by Drs. Yan Hong and Chengxin Yi (Yi et al. 2010). Seeds were germinated on ½ Murashige and Skoog salt medium. Cotyledons were harvested from 5-7 day-old seedlings, cut into small pieces (5×5 mm) and used as explants.

Jatropha Transformation Procedure:

The detailed transformation protocol was previously described (Mao et al. 2010). In brief, the protocol consisted of 4 steps. (1) Co-cultivation. Small cotyledons pieces were incubated with Agrobacterium cells harboring the target expression cassette in 20 ml of medium II for 10-20 min at 25° C. Explants were then transferred to the co-cultivation medium for 2-3 days at 22° C. in the dark. Then, the co-cultivation explants were rinsed several times with sterile water followed by one wash with 300 mgL⁻¹ cefotaxine. The cotyledon tissues were blotted dry on a pad of sterilized paper to remove excess surface water. The explants were then placed on a callus formation medium and transferred to darkness at 25±1° C. for 3 weeks. Under this condition, the un-transformed explants usually turned brown. (2) Shoot regeneration. Explants with newly emerged hygromycin-resistant callus were transferred into shoot regeneration medium I for 3 weeks at 25° C. under 16 h light (100 μmol m⁻² S⁻¹)/8 h dark cycles. During this period, any shoots regenerated from callus (about 35-40%) would be transferred to shoot regeneration medium II. Callus with no regenerated shoots were transferred to shoot regeneration medium III for further regeneration. (3) Shoot elongation. After 4 weeks, the regenerated shoots were transferred into shoot elongation medium for elongation and bud multiplication. (4A) Rooting. Elongated shoots of about 2.5 cm were rooted on a rooting medium. Normally, more than one month is required to obtain roots. (4B) Another alternative was to use grafting to increase the plant survival rate. Elongated shoots were used as scions for grafting onto non-transgenic rootstocks. Healthy and vigorously growing Jatropha plants were chosen to be rootstocks. Both scions and rootstocks were cut into the cambium region so that phloem tissues from both parts will connect after joining. The graft joint was then wrapped with parafilm and secured by a tape. The grafted Jatropha plants were maintained under low light fluences (10-20 μmol m⁻² S⁻¹) and 85% humidity for 7 days.

Transgenic Plasmids Construction and Materials:

Jatropha curcas Flowering locus T gene was firstly identified from a database of sequenced cDNA library prepared from J. curcas seeds. This cDNA fragment was PCR-amplified with forward primer 5′-ATAAGTCGACATGAGGGATCAATTTAGAGA-3′ (SEQ ID NO:20) and reverse primer 5′-TTATTTCTAGATCACCGTCTCCGTCCTCCGGT-3′ (SEQ ID NO:21). The PCR fragment was inserted in the sense orientation into the SalI/XabI sites of pCABMIA1300-3HA vector.

To generate the β-estradiol chemical-regulated inducible JcFT overexpression lines, we used a JcFT coding region fragment. This cDNA fragment was PCR-amplified with forward primer 5′-ATAACTCGAGATGAGGGATCAATTTAGAGA-3′ (SEQ ID NO:22) and reverse primer 5′-TTATTACTAGTTCACCGTCTCCGTCCTCCGGT-3′ (SEQ ID NO:23). The PCR fragment was inserted in the sense orientation into the XhoUSpeI sites of pX7-GFP vector as described previously (Guo et al. 2003). The construct was named as pX7-JcFT.

Plants were grown in a greenhouse under natural photoperiods and ambient temperature (ranged from 25-35° C.) in Singapore.

RNA Extraction, JcFT Cloning and Analysis:

100 mg leaf tissues were ground to fine powder in liquid N₂ and extracted with plant RNA purification reagent (Invitrogen, CA USA). RNA concentration was measured by Nanodrop (Thermo, DE, USA). M-MLV reverse transcriptase (Promega, WI, USA) was used for reverse transcription reactions and cDNAs production. The cDNAs were used to amplify FT coding region. Real-time PCR was performed with Power SYBR® Green PCR Master mix (Applied Biosystems, CA, USA) and run in ABI7900HT. All samples were run in triplicates and the data was analyzed with RQ manager at a pre-set Ct value (Applied Biosystems, CA, USA). Ct values included in the analyses were based on three biological replicates, with three technical replicates for each biological sample. Standard deviation was calculated based on the three biological replicates.

Southern Blot Analysis:

Total genomic DNA was isolated from the leaves of glasshouse-grown transgenic or control plants by the Cetyltrimethyl ammonium bromide (CTAB) method (Allen et al. 2006). Genomic DNA was digested with restriction enzymes and separated on 0.8% agarose gels. The gels were processed and transferred to a nylon Hybond-N⁺ membrane (GE Biosciences, USA) following standard procedures (Sambrook et al., 1989). Membranes were hybridized with HPT or JcFT ORF probes. The probes were labelled with [α-32P]-deoxycytidine triphosphate ([α-32P]-dCTP) by random prime synthesis using Amersham Rediprimer II Random Prime Labelling System (GE Biosciences, USA). Hybridization was performed overnight at 42° C. using the ULTRAHyb-Oligo hybridization buffer (Ambion, TX, USA) and signals were detected by autoradiography.

### Example 2

### Identified JcFT Coding Region and Genomic Sequence

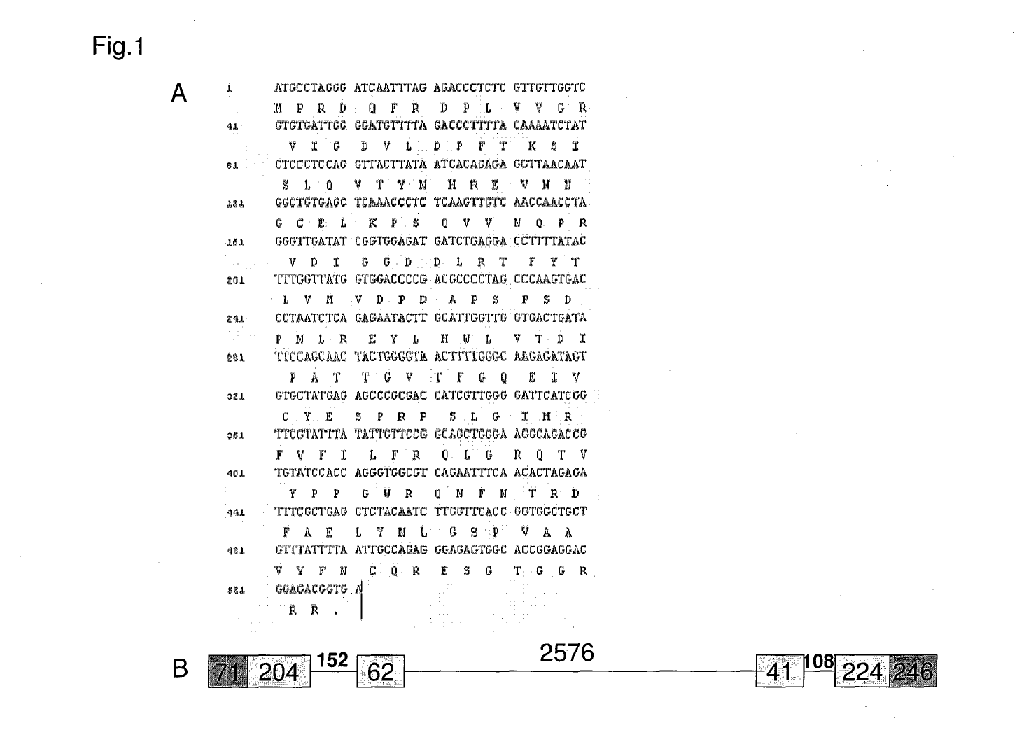

Jatropha curcas Flowering locus T gene was firstly identified from a database of sequenced cDNA library prepared from J. curcas seeds (FIG. 1A). The genomic sequence was identified from the deep sequencing database of Temasek Life Science labs (Dr. Hong Yan and Dr. Genhua Yue). The exon-intron genomic organization was manually identified (FIG. 1B). Box and line indicate exon and intron regions, respectively. Numbers showed the base pairs of nucleotides. Red box indicates 3′ or 5′ untranslated regions associated with the expression of the gene product.

To identify other Flowering locus T-like PEBP family genes from J. curcas, a similar strategy like the one described above was used to clone JcTFL1L-1, JcTFL1L-2, JcMFT-1 and JcMFT-2. Primers listed below were used. The PCR products were further cloned into pCAMBIA13-3HA vectors to generate pCAMBIA-35S:JcTFL1L-1, pCAMBIA-35S:JcTFL1L-2, pCAMBIA-35S:JcMFT-1, and pCAMBIA-35S:JcMFT-2.

| (SEQ ID NO: 24) | |
|---|---|
| TFL1L-2-F: | AAAGTCGACATGGAAAAACCAGTAGAC |
| (SEQ ID NO: 25) | |
| TFL1L-2-R: | AAATCTAGAGCGTCTTCTTGCAGCAGT |
| (SEQ ID NO: 26) | |
| TFL1L-1-F: | GAAGTCGACATGGCAAAAGTGTCAGAT |
| (SEQ ID NO: 27) | |
| TFL1L-1-R: | AAATCTAGAGCGTCTTCTTGCAGCAGT |
| (SEQ ID NO: 28 | |
| MFT-1-F: | AAAGTCGACATGGCGGCCTCTGTTGAT |
| (SEQ ID NO: 29) | |
| MFT-1-R: | GAATCTAGAAAACCTTTTGGCTGCTG |
| (SEQ ID NO: 30 | |
| MFT-2-F: | AAAGTCGACATGGCTCGCTCTCTT |
| (SEQ ID NO: 31) | |
| MFT-2-R: | GGGTCTAGAAACGTTTTTTAACTGC |

The coding sequence, amino acid sequence and genomic sequence for JcTFL1L-1 are set forth in SEQ ID NO:32, SEQ ID NO:33 and SEQ ID NO:34, respectively. The coding sequence, amino acid sequence and genomic sequence for JcTFL1L-2 are set forth in SEQ ID NO:35, SEQ ID NO:36 and SEQ ID NO:37, respectively.

### Example 3

### Transgenic Arabidopsis Overexpressing JcFT Showed Early Flowering Phenotypes

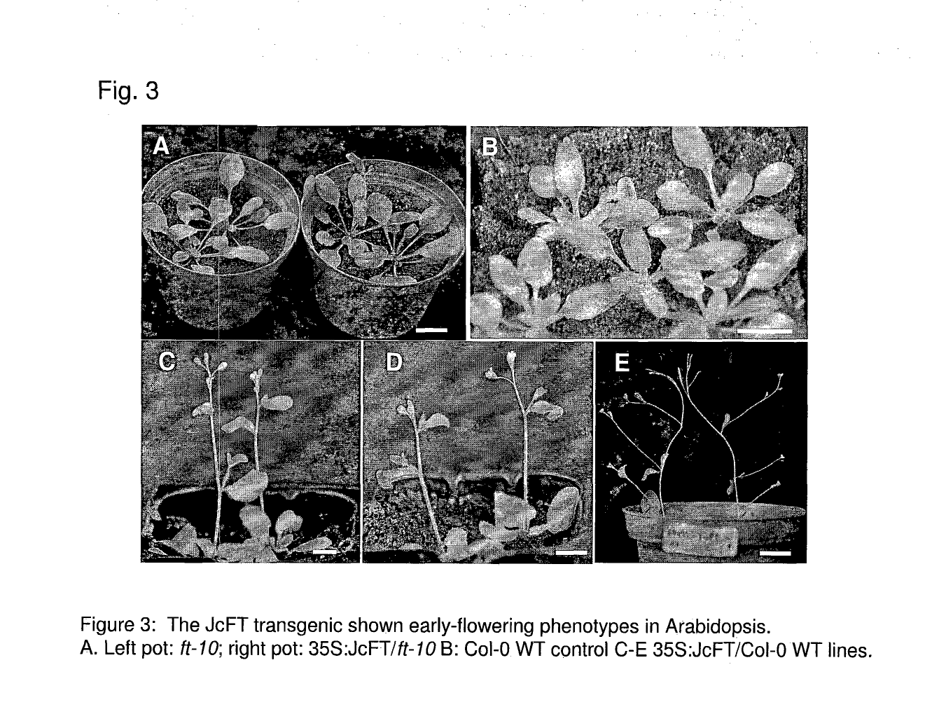

To assess the potential roles of PEBP family genes (JcFT, JcTFL1L-1, JcTFL1L-2, JcMFT-1 and JcMFT-2) in flowering time control, the putative JcFT gene (1300-3HA-JcFT) was overexpressed in Arabidopsis using a cauliflower mosaic virus 35S promoter. The 35S:JcFT construct was introduced into the late-flowering Arabidopsis ft-10 mutant plants (in the Col-0 background). At least 10 independent homozygous T3 lines were produced, and representative plants grown under LD conditions are shown in FIG. 3A. 35S:JcFT complemented the ft-10 mutation and caused plants to flower like in wild-type Col-0 plants. These results suggest that JcFT can functionally complement the ft-10 mutation and its expression results in very early flowering when driven by the 35S promoter.

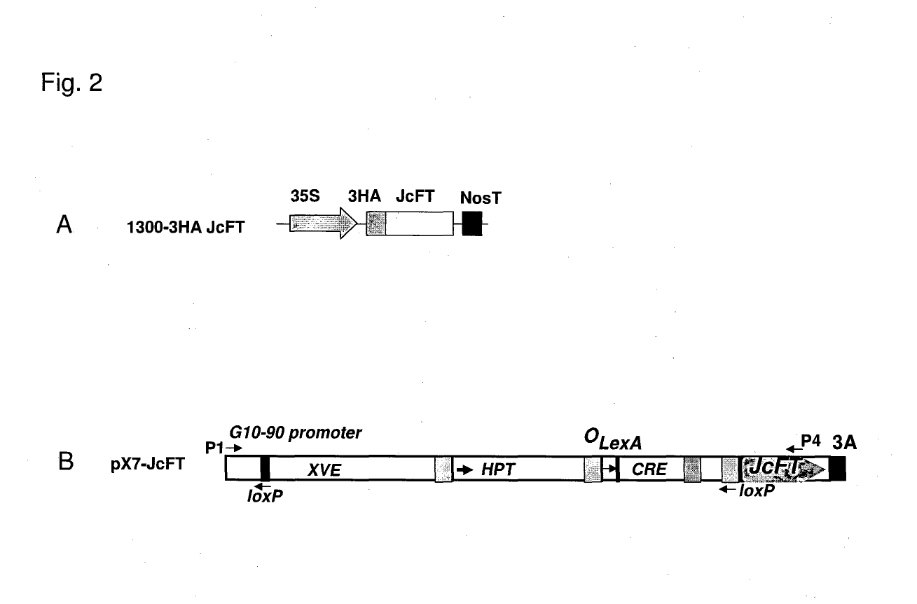

Flowering dipping method was further used to obtain transgenic Arabidopsis lines which overexpressed JcFT (1300-3HA-JcFT, FIG. 2A) in WT Col-0 background. Early flowering phenotypes were easily observed in T1 plants (FIG. 3B-3E) compared with WT Col-0 control plants (FIG. 3A and Table 1).

TABLE 1
Flowering Time of Transgenic Arabidopsis Lines Overexpressing JcFT

| | | Bolting | Total Leaf | Time of | | |
|---|---|---|---|---|---|---|
| | No. of | Time | No. formed | Anthesis | Rosette | Cauline |
| Line | Plants | (days) | at Bolting | (days)* | Leaf | Leaf |
| Wt(Col-0) | 40 | 23.88 ± 1.92 | 10.63 ± 1.44 | 27.5 ± 1.92 | 11.38 ± 1.29 | 2.1 ± 0.63 |
| | 21 | | | | | |
| 35S:JcFT-wt | 21 | | | | | |
| #3 | 21 | 18.95 ± 1.16 | 7.52 ± 0.75 | 22.71 ± 2.51 | 7.76 ± 0.77 | 1.9 ± 0.77 |
| #5 | 21 | 19.14 ± 1.20 | 8.90 ± 1.22 | 25.48 ± 1.40 | 9.10 ± 1.00 | 2.19 ± 0.60 |
| #6 | 21 | 21.33 ± 1.35 | 8.81 ± 0.75 | 26.67 ± 1.20 | 9.62 ± 1.20 | 1.86 ± 0.57 |
| #8 | 21 | 20.10 ± 1.73 | 8.67 ± 0.91 | 25.29 ± 1.49 | 9014 ± 1.11 | 2.19 ± 0.51 |
| #9 | 21 | 18.24 ± 0.62 | 7.48 ± 0.51 | 20.90 ± 0.30 | 7.00 ± 0.32 | 2.14 ± 0.57 |
| #13 | 21 | 21.38 ± 1.24 | 9.24 ± 0.83 | 26.81 ± 1.81 | 10.00 ± 1.18 | 2.38 ± 0.67 |
| #15 | 21 | 17.19 ± 0.51 | 6.90 ± 0.62 | 21.10 ± 0.30 | 6.81 ± 0.51 | 1.76 ± 0.44 |
| #16 | 21 | 17.10 ± 0.30 | 7.10 ± 0.54 | 21.05 ± 0.22 | 7.24 ± 0.62 | 1.81 ± 0.51 |
| #20 | 21 | 20.90 ± 1.18 | 9.29 ± 0.96 | 25.14 ± 1.15 | 9.81 ± 0.98 | 1.86 ± 0.57 |

*Time of anthesis is the day when the floral organ became visible.

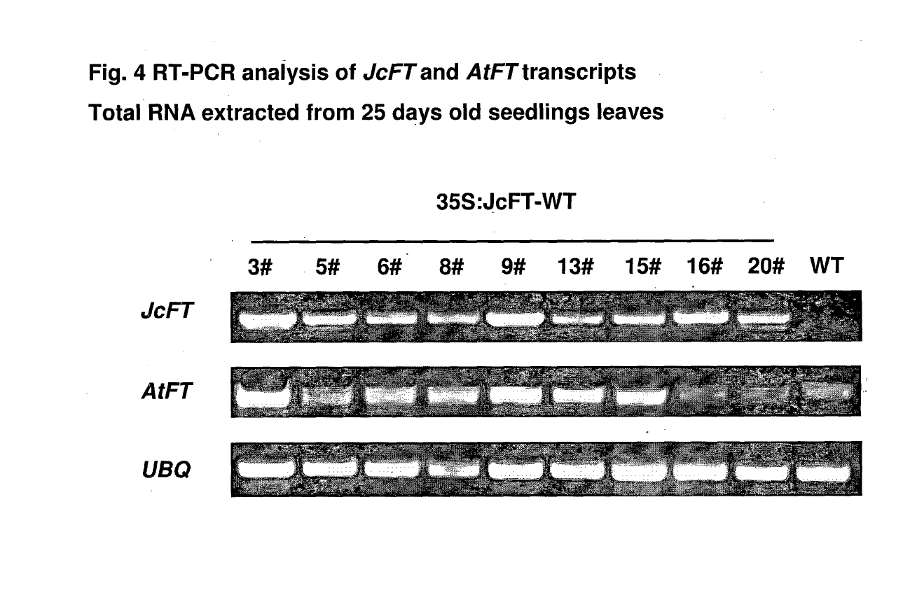

Molecular analysis of the JcFT overexpressing transgenic Arabidopsis suggested almost all of these lines expressed high levels of JcFT mRNA (FIG. 4). JcFT overexpressing Arabidopsis lines produced more lateral branches (FIG. 3E)

### Example 4

### Overexpression of JcFT Strongly Accelerates Flowering in Transgenic Jatropha Plants

To explore the effect of JcFT overexpression on flowering time in Jatropha plants, elite Jatropha JcMD-44 was transformed with the same 35S:JcFT construct that was used to complement the Arabidopsis ft-10 mutant.

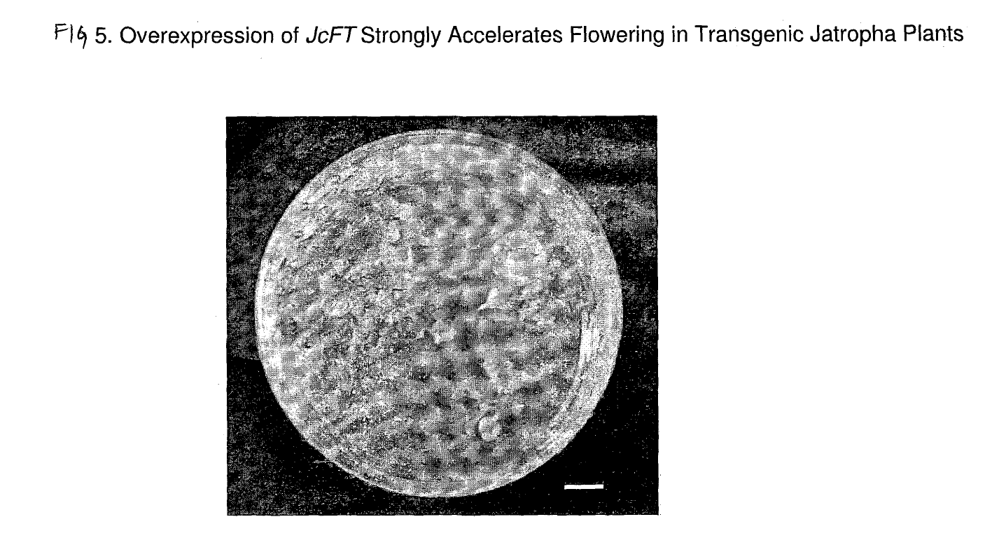

Detailed Jatropha transformation protocol was previously described (Mao et al. 2010). Shoots were regenerated directly from explants. Two months after co-culture with Agrobacterium tumefaciens strain, which harbored the 35S:FT construct, almost all of regenerated hygromycin resistant shoots began to develop inflorescence (FIG. 5). The strong 35S promoter generated too high a level of JcFT, which caused early flowering even in young shoots just regenerated from calli.

### Example 5

### Early Flowering Phenotypes in JcFT Overexpressing Jatropha

To accelerate Jatropha flowering time and the same time to maintain other traits normal, elite Jatropha JcMD-44 was transformed with a weak promoter (G10-90) driven pX7-JcFT, which resulted in the inducible expression of JcFT.

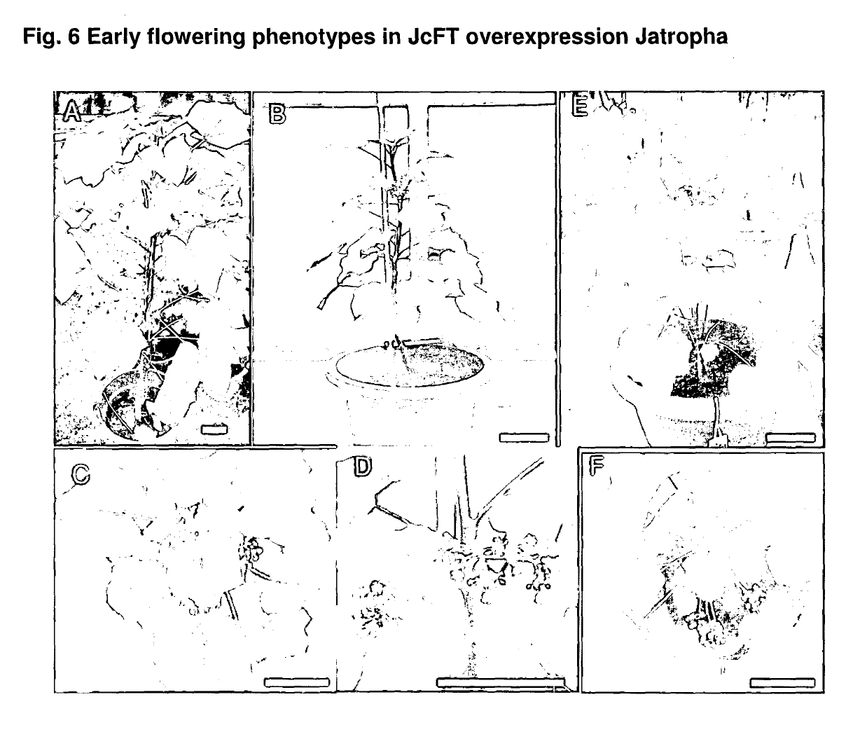

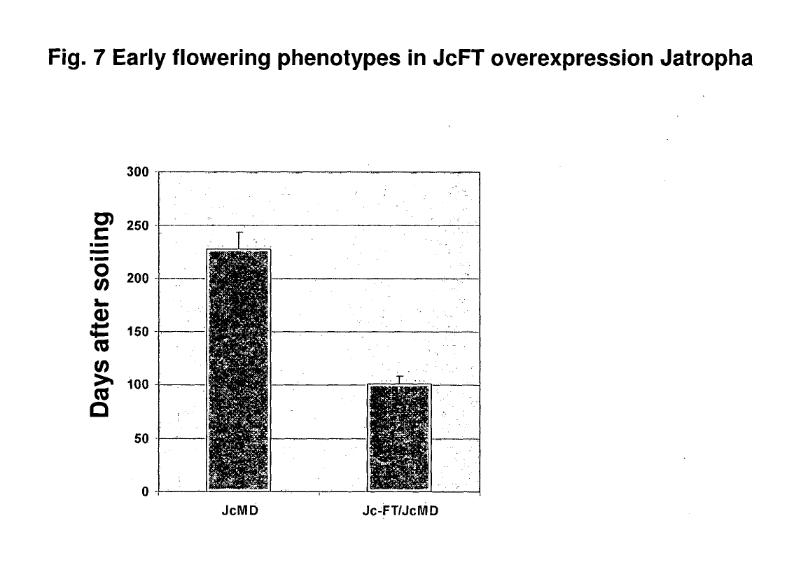

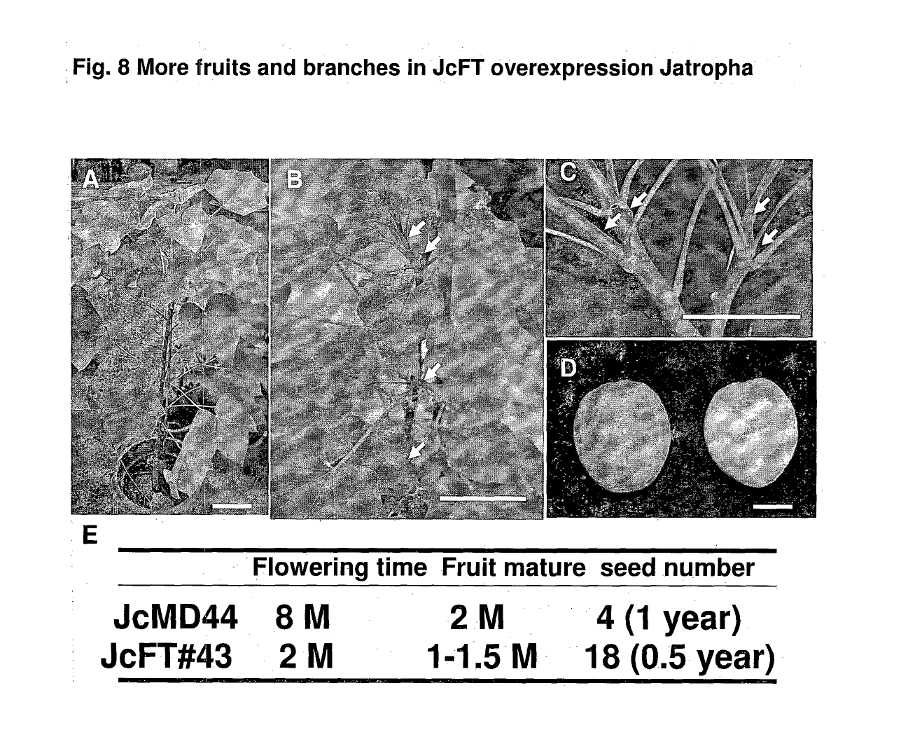

The flowering time of T0 plants (plants regenerated directly from explants) was measured. Ten independently generated pX7-JcFT plants were obtained, and all flowered significantly earlier than the seed-germinated untransformed plant controls (FIG. 6). Under greenhouse conditions in Singapore, it takes 7-8 months for elite JcMD plants to flower from seed germination and an additional 2 months is required to obtain seeds. By contrast, it only took an average of 101 days for JcFT overexpressing T0 plants to flower (FIG. 7). Moreover, these transgenic JcFT overexpressing lines produced more branches and fruits (FIG. 8).

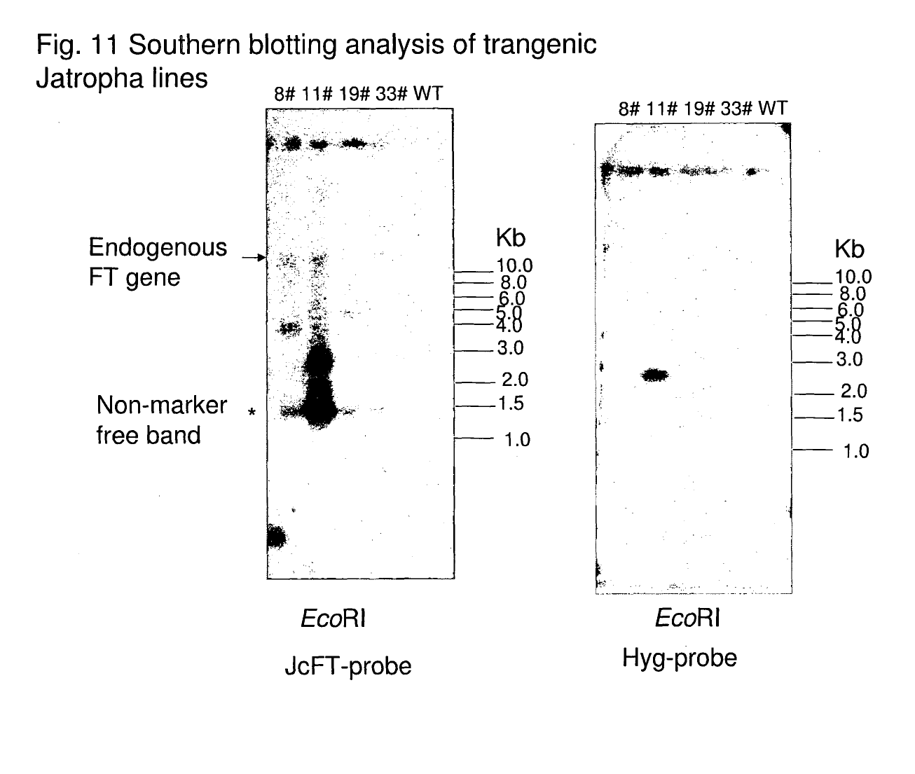

Molecular analysis of the JcFT overexpressing transgenic Jatropha suggested most of these lines expressed higher levels of JcFT mRNA. Southern blots were used to examine the transgenic Jatropha lines. There was only one endogenous FT gene locus (FIG. 11).

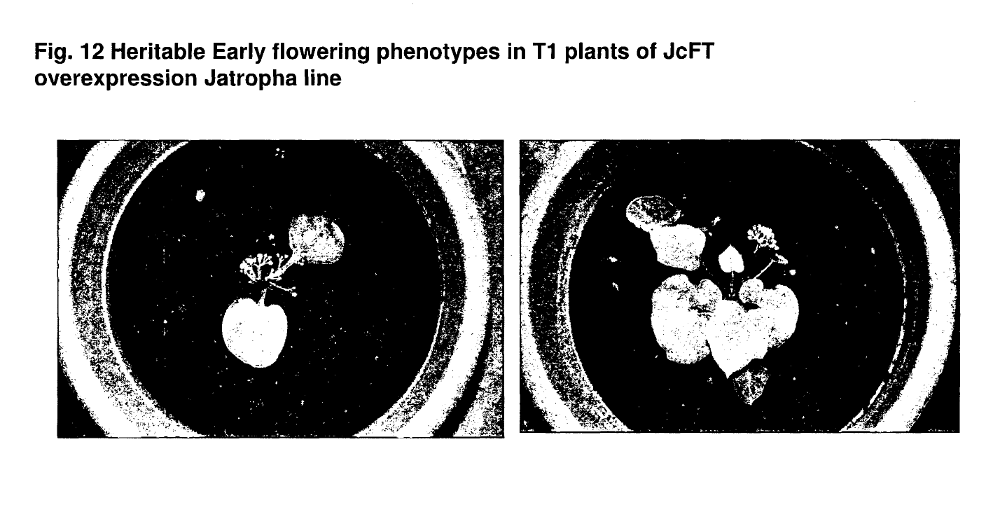

Heritable early flowering phenotypes were observed in many T1 plants of JcFT overexpressing Jatropha line (FIG. 12).

### Example 6

### Early Flowering Phenotypes in JcTFL1L-1 and JcTFL1L-2 Overexpressing Arabidopsis

To investigate whether the Euphorbiacious PEBP homologs exhibit a conserved function in the regulation of flowering time, we cloned each coding region downstream of the constitutive CaMV ³⁵S promoter and transformed the obtained gene cassettes into Arabidopsis. Ectopic expression of JcFT resulted in an extremely early-flowering phenotype with 6.9 leaves (including cotyledons) formed prior to flowering (FIGS. 3C, D, E). The late-flowering phenotype of the ft-10 mutant was fully complemented by the ectopic expression of JcFT (FIG. 3A). This suggests that JcFT represents a true FT homolog in Jatropha curcas.

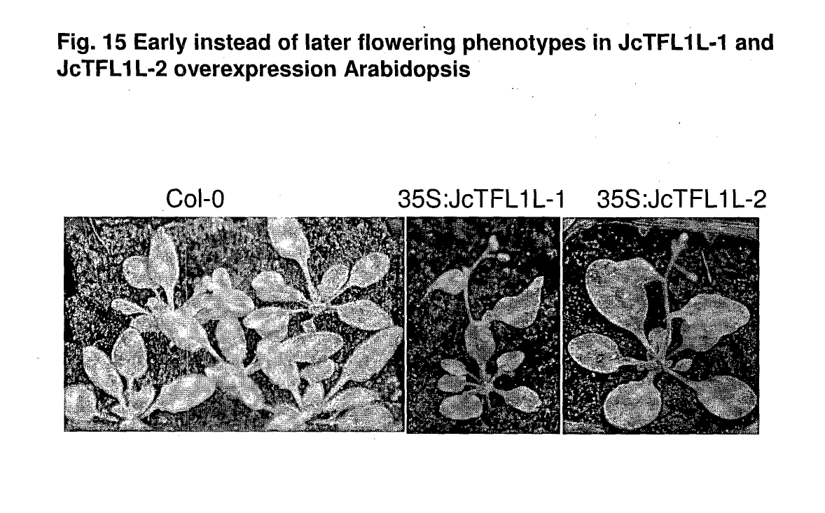

Surprisingly, transgenic lines expressing JcTFL1L-1 and JcTFL1L-2 displayed an early instead of a very late-flowering phenotype as was described for Arabidopsis transgenic lines overexpressing TFL1. Both JcTFL1L-1 and JcTFL1L-2 promoted flowering although their activity was weaker with an average of 2 leaves fewer than those of wild-type at the time of flowering (FIG. 15 and Table 2). The plants were not merely early-flowering, like FT overexpressing line, but they also displayed some morphological features of plants overexpressing FT, such as slimmer inflorescences (FIG. 15). The latter features were previously observed in Arabidopsis plants overexpressing JcFT (FIGS. 3C, D and E), a well-known flowering promoters.

TABLE 2
Flowering Time of Transgenic Arabidopsis
Lines Overexpressing JcTFL1L-1 and JcTFL1L-2

| | | Total | Time of | | |
|---|---|---|---|---|---|
| | No. | Leaf No. | Anthesis | Rosette | Cauline |
| Genotype | Plants | (bolting) | (days) | Leaves | Leaves |
| WT | 40 | 10.6 ± 1.4 | 27.5 ± 1.9 | 11.4 ± 1.3 | 2.1 ± 0.6 |
| 35S:JcTFL1L-1 | 15 | 8.4 ± 0.7 | 16.2 ± 1.5 | 9.9 ± 1.3 | 1.9 ± 0.5 |
| 35S:JcTFL1L-2 | 15 | 8.5 ± 0.9 | 16.5 ± 1.2 | 9.8 ± 1.1 | 1.5 ± 0.5 |
| 35S:JcFT #15 | 21 | 6.9 ± 0.6 | 21.1 ± 0.3 | 6.8 ± 0.5 | 1.8 ± 0.4 |

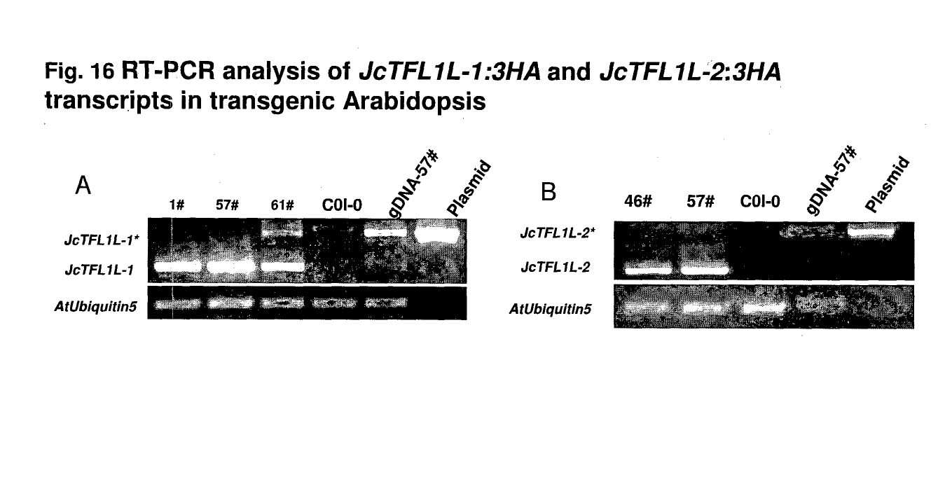

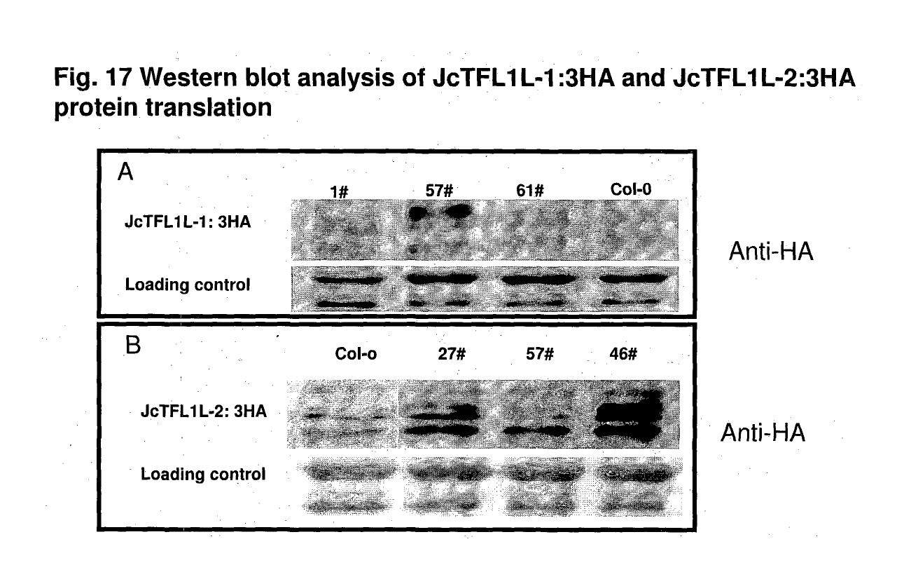

Further molecular analysis on RNA level (FIG. 16) and protein level (FIG. 17) suggested that JcTFL1L-1 and JcTFL1L-2 act like a flowering promoter rather than a repressor, despite the fact that both of them cluster in the TFL1-like clade of the PEBP family.

### Example 7

### Transgenic Jatropha with JcTFL1L-1 Overexpression Showed Early Flowering and Self-pruning Phenotypes

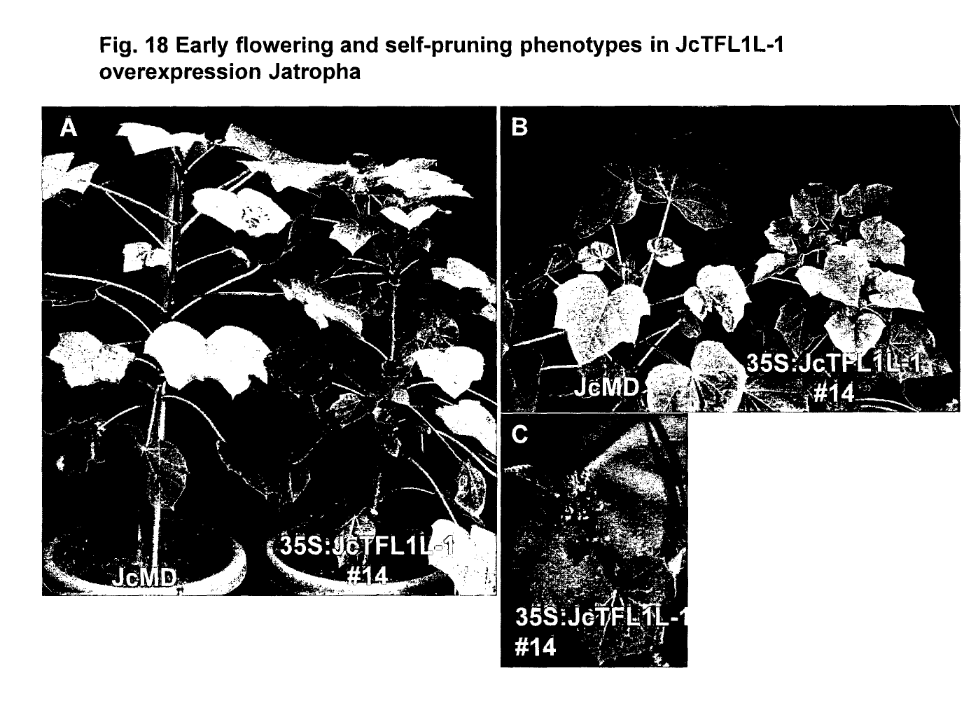

With the transformation method described before (Mao et al., 2010; Qu et al., 2012)), we generated transgenic Jatropha which overexpresses either JcTFL1L-1 or JcTFL1L-2. Transgenic Jatropha overexpress JcTFL1L-1 overexpression showed early flowering and self-pruning phenotypes (FIG. 18) and Table 3.

TABLE 3
Flowering Time for Transgenic Jatropha Lines with JcTFL1L-1
Overexpression

| Genotype | No. plants | Days after soiling |
|---|---|---|
| JcMD | 8 | 223 ± 12.3 |
| 35S:JcTFL1L-1/JcMD | 2 | 145 ± 5 |

There is average of 7 shoots in JcTFL1L-1 overexpression transgenic Jatropha, on contrast of 1 shoot in WT JcMD plants (FIG. 18).

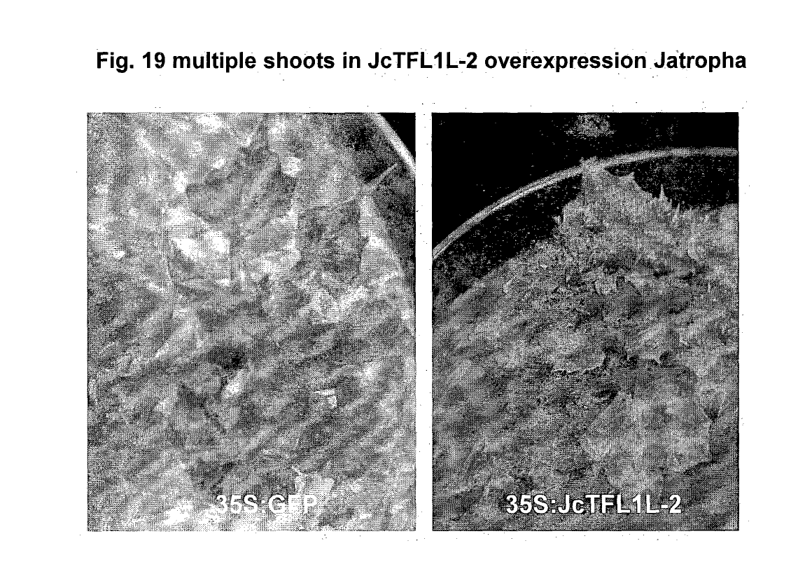

For JcTFL1L-2 overexpression transgenic Jatropha, there are many young shoots in shoot regenerating step during transformation process (FIG. 19), indicating a role of JcTFL1L-2 in meristem size determination controlling by WUSCHEL which regulates the maintenance of stem cell populations in meristem region in Arabidopsis (Ikeda et al., 2009).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Abdullah, R. et al. (2005). Immature embryo: A useful tool for oil palm (Elaeis guineensis Jacq.) genetic transformation studies. Electronic Journal of Biotechnology [online] vol. 8, no. 1 [Apr. 15, 2005). Available from: http colon//www dot ejbiotechnology dot info/content/vol8/issue1/full/1/index.html.
Achten, W. et al. (2010). Towards domestication of Jatropha curcas: a review. Biofuels 1:91-107.
Allen, G. C. et al. (2006). A modified protocol for rapid DNA isolation from plant tissues using cetyltrimethylammonium bromide. Nature Protocols 1:2320-2325.
Blackman, B. K. et al. (2010). The role of recently derived FT paralogs in sunflower domestication. Curr Biol 20:629-635.
Carmel-Goren, L. et al. (2003). The SELF-PRUNING gene family in tomato. Plant Mol Biol 52:1215-1222.
Chiu, W. et al. (1996). Engineered GFP as a vital reporter in plants. Current Biology 6:325-330.
Chowdhury, M. K. U. et al. (1997). Evaluation of five promoters for use in transformation of oil palm (Elaeis guineensis Jacq.) Plant Cell Reports 16:277-281.
Christensen, A. H. and Quail, P. H, (1989). Sequence analysis and transcriptional regulation by heat shock of polyubiquitin transcripts from maize. Plant Mol Biol 12:619-632.
Christensen, A. H. et al. (1992). Maize polyubiquitin genes: structure, thermal perturbation of expression and transcript splicing, and promoter activity following transfer to protoplasts by electroporation. Plant Mol Biol 18:675-689.
De Wet, J. R. et al. (1987). Firefly luciferase gene: structure and expression in mammalian cells. Mol Cell Biol 7:725-737.
Goff, S. A. et al. (1990). Transactivation of anthocyanin biosynthetic genes following transfer of B regulatory genes into maize tissues. EMBO J 9:2517-2522.
Guo, H. S. et al. (2003). A chemical-regulated inducible RNAi system in plants. Plant J 34:383-392.
Ikeda, M. et. al. (2009). Arabidopsis WUSCHEL is a bifunctional transcription factor that acts as a repressor in stem cell regulation and as an activator in floral patterning. Plant Cell 21:3493-3505.
Imaizumi, T. (2010). Arabidopsis circadian clock and photoperiodism: time to think about location. Curr Opin Plant Biol 13:83-89.
Jefferson, R. A. et al. (1991). Plant Molecular Biology Manual, ed. Gelvin et al., Kluwer Academic Publishers, pp. 1-33.
Kain, S. R. et al. (1995). Green fluorescent protein as a reporter of gene expression and protein localization. Biotechniques 19:650-655.
Kobayashi, Y. and Weigel, D. (2007). Move on up, it's time for change—mobile signals controlling photoperiod-dependent flowering. Genes & Develop 21:2371-2384.
Kramer, W. and Fritz, H.-J., (1987). Oligonucleotide-directed construction of mutagenesis via gapped duplex DNA. Methods in Enzymology 154:350-367.
Last, D. I. et al. (1991). pEmu: an improved promoter for gene expression in cereal cells. Theor Appl Genet 81:581-588.
Lifschitz, E. et al. (2006). The tomato FT ortholog triggers systemic signals that regulate growth and flowering and substitute for diverse environmental stimuli. Proc Natl Acad Sci USA 103:6398-6403.
Mao, H. Z. et al. (2010). Genetic transformation of Jatropha curcas. International Publication No. WO 2010/071608.
Masli, D. I. A. et al. (2009). Transformation of oil palm using Agrobacterium tumefaciens. J Oil Palm Res 21:643-652.
McElroy, D. et al. (1990). Isolation of an efficient actin promoter for use in rice transformation. Plant Cell 2:163-171.
Odell, J. T. et al. (1985). Identification of DNA sequences required for activity of the cauliflower mosaic virus 35S promoter. Nature 313:810-812.
Omidvar, V. et al. (2008). A transient assay to evaluate the expression of polyhydroxybutyrate genes regulated by oil palm mesocarp-specific promoter. Plant Cell Rep 27:1451-1459.
Parveez, G. K. A. et al. (2000). Transgenic oil palm: production and projection. Biochemical Society Transactions 28:969-972.
Parveez, G. K. A. (2008). Biolistic mediated production of transgenic oil palm. Methods Mol Biol 477:301-320.
Pin, P. A. et al. (2010). An antagonistic pair of FT homologs mediates the control of flowering time in sugar beet. Science 330:1397-1400.
Qu, J. et al. (2012). Development of marker-free transgenic Jatropha plants with increased levels of seed oleic acid. Biotechnol Biofuels 5:10.
Srikanth, A. and Schmid, M. (2011). Regulation of flowering time: all roads lead to Rome. Cell Mol Life Sci 68:2013-2037.
Velten, J. et al. (1984). Isolation of a dual plant promoter fragment from the Ti plasmid of Agrobacterium tumefaciens. EMBO J 3:2723-2730.
Yi, C. et al. (2010). Does epigenetic polymorphism contribute to phenotypic variances in Jatropha curcas L.? BMC Plant Biol 2010 10(259).

1. An isolated nucleic acid encoding a protein selected from the group consisting of
a JcTFL1L-1 protein having the amino acid sequence set forth in SEQ ID NO:33,
a protein having at least 90% identity to the amino acid sequence set forth in SEQ ID NO:33 and having the flower promoting activity of the JcTFL1L-1 protein,
a JcTFL1L-2 protein having the amino acid sequence set forth in SEQ ID NO:36,
a protein having at least 90% identity to the amino acid sequence set forth in SEQ ID NO:36 and having the flower promoting activity of the JcTFL1L-2 protein,
a JcFT protein having the amino acid sequence set forth in SEQ ID NO:2, and.
a protein having at least 90% identity to the amino acid sequence set forth in SEQ ID NO:2 and having the flower promoting activity of the JcFT protein.

2. The isolated nucleic acid of claim 1 selected from the group consisting of
a nucleic acid encoding the JcTFL1L-1 protein, wherein the nucleic acid has the nucleotide sequence set forth in SEQ ID NO:32,
a nucleic acid encoding the JcTFL1L-2 protein, wherein the nucleic acid has the nucleotide sequence set forth in SEQ ID NO:34, and
a nucleic acid encoding the JcFT protein, wherein the nucleic acid has the nucleotide sequence set forth in SEQ ID NO:1.

3. The isolated nucleic acid of claim 1 which further comprises a plant operable promoter operably linked to the nucleic acid.

4. An expression vector comprising the isolated nucleic acid of claim 1.

5. A transgenic plant cell, plant or plant seed comprising the isolated nucleic acid of claim 1 stably integrated into its genome.

6. The transgenic plant cell, plant or plant seed of claim 5, wherein the plant is a Cassava (Manihot esculenta), a Castor oil plant (Ricinus communis), a tung tree (Vernicia fordii), a physics nut (Jatropha curcas), or a rubber tree (Hevea brasiliensis).

7. The transgenic plant cell, plant or plant seed of claim 5, wherein the plant is Jatropha.

8. A method for producing a transgenic plant which comprises introducing the isolated nucleic acid of claim 1 or an expression vector comprising the isolated nucleic acid of claim 1 into a plant, wherein the transgenic plant has the nucleic acid stably integrated in its genome.

9. A method for producing a transgenic plant which comprises transfecting the isolated nucleic acid of claim 1 or an expression vector comprising the isolated nucleic acid of claim 1 into a plant cell or plant cells and regenerating a transgenic plant from the transfected plant cell or transfected plant cells, wherein the transgenic plant has the nucleic acid stably integrated in its genome.

10. The method of claim 8, wherein the plant is a Cassava (Manihot esculenta), a Castor oil plant (Ricinus communis), a tung tree (Vernicia fordii), a physics nut (Jatropha curcas), or a rubber tree (Hevea brasiliensis).

11. The method of claim 8, wherein the transgenic plant is Jatropha.

12. A method of promoting the flowering time of a plant which comprises introducing the nucleic acid of claim 1 or an expression vector comprising the isolated nucleic acid of claim 1 into a plant, wherein the isolated nucleic acid is expressed in the plant thereby promoting the flowering time of the plant.

13. A method of promoting the flowering time of a plant comprising transfecting the nucleic acid of claim 1 or an expression vector comprising the isolated nucleic acid of claim 1 into a plant cell or plant cells and growing a plant from the transfected plant cell or transfected plant cells, wherein the isolated nucleic acid is expressed in the plant thereby promoting the flowering time of the plant.

14. The method of claim 12, wherein the plant is a Cassava (Manihot esculenta), a Castor oil plant (Ricinus communis), a tung tree (Vernicia fordii), a physics nut (Jatropha curcas), or a rubber tree (Hevea brasiliensis).

15. The method of claim 12, wherein the plant is Jatropha.

16. An isolated protein selected from the group consisting of
a JcTFL1L-1 protein having the amino acid sequence set forth in SEQ ID NO:33,
a protein having at least 90% identity to the amino acid sequence set forth in SEQ ID NO:33 and having the flower promoting activity of the JcTFL1L-1 protein,
a JcTFL1L-2 protein having the amino acid sequence set forth in SEQ ID NO:36,
a protein having at least 90% identity to the amino acid sequence set forth in SEQ ID NO:36 and having the flower promoting activity of the JcTFL1L-2 protein,
a JcFT protein having the amino acid sequence set forth in SEQ ID NO:2, and.
a protein having at least 90% identity to the amino acid sequence set forth in SEQ ID NO:2 and having the flower promoting activity of the JcFT protein.

17. An expression vector comprising the isolated nucleic acid of claim 3.

18. A transgenic plant cell, plant or plant seed comprising the isolated nucleic acid of claim 3 stably integrated into its genome.

19. A method for producing a transgenic plant which comprises introducing the isolated nucleic acid of claim 3 or an expression vector comprising the isolated nucleic acid of claim 3 into a plant, wherein the transgenic plant has the nucleic acid stably integrated in its genome.

20. A method for producing a transgenic plant which comprises transfecting the isolated nucleic acid of claim 3 or an expression vector comprising the isolated nucleic acid of claim 3 into a plant cell or plant cells and regenerating a transgenic plant from the transfected plant cell or transfected plant cells, wherein the transgenic plant has the nucleic acid stably integrated in its genome.

21. A method of promoting the flowering time of a plant which comprises introducing the nucleic acid of claim 3 or an expression vector comprising the isolated nucleic acid of claim 3 into a plant, wherein the isolated nucleic acid is expressed in the plant thereby promoting the flowering time of the plant.

22. A method of promoting the flowering time of a plant comprising transfecting the nucleic acid of claim 3 or an expression vector comprising the isolated nucleic acid of claim 3 into a plant cell or plant cells and growing a plant from the transfected plant cell or transfected plant cells, wherein the isolated nucleic acid is expressed in the plant thereby promoting the flowering time of the plant.


SEQUENCE LISTING (37 sequences)

SEQ ID NO: 1 - DNA, 531 bp, Jatropha curcas
  CDS (1)..(528)
atgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggttatggtggaccccgacgcccctagcccaagtgaccctaatctcagagaatacttgcattggttggtgactgatattccagcaactactggggtaacttttgggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtga

SEQ ID NO: 2 - protein, 176 aa, Jatropha curcas
MPRDQFRDPLVVGRVIGDVLDPFTKSISLQVTYNHREVNNGCELKPSQVVNQPRVDIGGDDLRTFYTLVMVDPDAPSPSDPNLREYLHWLVTDIPATTGVTFGQEIVCYESPRPSLGIHRFVFILFRQLGRQTVYPPGWRQNFNTRDFAELYNLGSPVAAVYFNCQRESGTGGRRR

SEQ ID NO: 3 - DNA, 17383 bp, Jatropha curcas
tgggcaaggcaagaaattggaagtgccttttttttttttttatgaacaaacataagctggttggtgtcagctggaatgcatcatagtttatcccatcgcccatcaccatgggaatcccaatattccccatgttttattttcttttttttttttgtaatattaaacttgtaatgattcagaagaaaaaagaaaccgaaacatatgttagtgttggttaatatagtacgtattgcaatatacatcagctggttggtgtctttttgtttccattttcctttgtaatctttgaaacgttcagacagcatatacctaatatatgtttccttccttgaaagacatttaatcagacatggccaagccaaagaggaagcatacaaattatttatgttggcgacgacgactccataatcaaattcaacaatcattattatttattagtattatgaaaaataattctctgtgttttttatgaaaagaagcccctcttattagttatgtacaggttgcggttttattttttttgtatctgcagctggcttttcctacttgcttcagctattctctaataataacaaatttatatttgctgataaaaaataaaaataaaaaataaacaataaaaaagactgtcgttatcagcaagggctactgatcacatgcgccctctcctcttcccatatcagcaacagcaagggctactgatcacatgtgtcctctcctcttccaaaatcttttttgtttctcctcttttaaactttgaatctgagactgtttgtcaatggaaagataattttattccttatactgaaaaagttaaacaaaaaaagataaatatatatgttctttaatcaaaatcagattatatgcatttatttccttacagcagccttacgtctctgcttgtacagacaacgacggaactatgaggtggccagcaccatcagtagcttccgcccctctagttttaaaatattttataaaacttaccttataaaacatatatagaaagaaaattttacatatcatctataaaattgtaattactgcccctaaatgtctatctagcagacccccgctaactcgagtgagcattcaaatatctgtctggttctgcagacttaattacttttaaataaagaacaaatgggagattttataaaaattctcttggtttagatttagttatggtggccgacatggatgtatgaagacggcaggcaatcccagaaaggaaaaagcaataagttgtaatctgcacttttgctgggatatttgtgttgaactccatctatcctaatcaagttttaattaattaatgaagctatgaacatattgacaggttgcagtgacgtgcaaaaggagggaagatatttttgattcaaagattctgcagtgaaaacgggaattgaaaaataaaaagaaattgagttgagttgggttggcatcaacgataaagctaaaaggtgtagtattcttctatacaaggaagattcttgtacaataaacattttgttctttctgttccaaataacaaatttcagaaacaaaaccaagccaaaagtactaccatccagtccactttcttactttcatcttcgaatgaatccaaaacgtgcacagccacaaaacatgatatgaaatatctgctaacattcagaaaaaatatgagccatgtatcctgagagttatgggtccccatccccatccccatgcttgcttttcggacattgataaggtacattatacaataatgtcttgtgccttgaacgtagcaggtttcagggtagatattagcttgacaagtggcacccaaaagaaagaatttcttttttttgtttttttctgcttcgaatgagttgagaaacggcaatgtggctcaatataacccacgaaattggtatattttccttgtttgaaatgaaatgattcggggtaagaattcaattcccgcctccttagtagaatatttatcaatttactgactttgttttatcaagtaatacaagattattgtattttggcgcaatatatatgtacgtaattgttatgaaattgcaattctacttaatgtgatgatttaattattttaatatcataaatatgaaattaataggataaattggaatactcgtgcttactatttttctcgtcttgacctgagtatatacttatgtgtagatacgtagtgaatatatgcatatacatgtagtgtagcgttgttttcttctgctttaaaggtctccttttctatcctatcactgtccacttcagaaaattaacccatacatttttttgccatttcttagttttttttttaacgtaaatgaataaaatgcaaaatgcaaaaaataaaaatataaagaactagatacccgcaatataaagtaaattctttcggtataaaggaggagaaaaacaaaaacaaaagaaggacatatggaagcttcattacaaaaatcagttgagggtaatttcgtagtatgcaaatcaacttcccgataagtaaattgatcttctattataaattattataatatccataatataaatgtattttcctcaactatttttaaaaaacttacttttttttatgcaagtgaagttggatgatttacttttcgagagtagaattgataatacaaactaacataagtattttatgtattttccaagaatttacatgtccttaatctacttaatctcccgaccaaacatcctagaagaatttgtatattgtttgttgttgggacgtgtatatgtaattatgtatctatgcatggaggacataaattaacatgcacacatttttgccgcctcaacttgaacaagcaaaatctcagcatctagacactgcggtctgtttgaatatcacctcttaatttatatttatttctgtccaagcatgaaaaaacaagcataatggaaaccaggatagataaattcggtgattctatgcatgttcgctaatcagtttcaaaaactgaacaccacctcgatattataaaaaaaaaaaaaaagagtaaatttgaattttactttaacagttgagtaattagggggtttaattctcatcttagtttgtacgcaccgaaaactagcttatcaagaaagctagcatatcattcatctttgcatcctgtactccgcacatggctatatatgagttcaaaacggaataagaaacaggggtccacttcacattctcaccatagttttagtgtaaattattatttacgctgtgaggttagactgaattaacgatttcatctctgtaattttgtaaaattattattgaactattcatcaacaaaaaaaaaaaaaaatagtcaacttgtaaagcaacatttctctcccctccgccctcaaccgatgctcccaaataacatcgaacattaaatatatctgcattttttttttatttgcgtgagtttgacttacgtttaatacaaaatgatttaataagttggattgattcaacttactcattttatttcatttcttactttatatttataagctacaattttaattaatctccatggaaaatccatattcttatttgttatcaaggccagtaaacgactcttcctacgttaaacccttgaatggttaaggaaatcatcaaacaaaataaattttatttccatatcctttagaacaatgaaatatgtagaggaattattaacctatgatttgtgagttcatctccattttattgtgtaaatggtgggggaaggagacagaatttatgtttgggagttggctaagaacggaggcggaggaggagattttttgttatcgatgtatagttttaaatttataatataattaaaaatgtatgaagcaatgatacatataatgactcactcttaatataattaaggcacgtgatgccattgcagaatttaattaaactaatttttttaaaatgtgcatttcatgttatttgtactcgttcatttttataaattacaagtaaatgtatgttacaaggtttttcatttctatctttatcgacggtggtttatgtaacttaatttaattttttcaactctcatgaaatgtcctcactcgcttattttatgttaatttaatttaataaaaaatctcaataaattaccaatacatatttatctttcatacacctacacactagctattcttatccgtttcttatatgcattatctatctagaacggaacttcctgctgagcgattgtagttcctagtaaaaaaagagcaagatacaagaatggaccagtggttgaaacggtgacgtagcgatcaagaataatattagaatgggaggcagtagattttgaagcggtaaattgagggagacgaaaatggtgaagatagtaatcttttccattttccagtatcacaagttataatttatacttttatattttttattttaaatatactcattcatctatctcaattatacctatctttctttcctttatatgtggaccccgtatcgtacattttacctttataatattctcatacctcggaatgttttgttccatccatatttatcaatgttataataaaaatttaatatgattgtaaaaaaaatgaataactataacgagtactaatgtgcaatgcacgatttgtaatttataatatatatacaagtgggaatgaaaaggcgaacattgaggctacaaaaaataaccctactaagtttatatatcgataaaaatatcattaaagattttagttttattaattggaaaataaagttgatttaattttgattaattatattaattttctatttaaatctaaattctattatataaaataataaattatattctatagtacataaggtattattaaatatagtaaaaatataatatctttaaggattaattttaattttattggttgaaaaaaatataatttttaattttagtcggttatattatattatgtaaggtgatttaatattaattgattatattatattataataggtatgtattaaatatgataagaatataatatatttaattattatttttaataatttcagtttgattggttataattatttcttacaaataacttattttacaaagaatatgtgtaatgtattattatttattaagcaatatattataaaatttatcatttgatataaaatagaaaataaaactcactagaaatatatataaaatataaaattatgatataaaaacattatgttagaaaaccatattacacattgaggattttttcttccaaaaaagcaatcatagaatttagcaaaagtgaaaaaaaagtaaatatatatatatagtattctaaaataatgattaacatattaattattataataatttaatattgatttcttatattttagtaatactagtcttctttatgccatgattgtacattgattatattcattatatatttatttttatactcgtttaattttttatttatataatattataatataatcttttatttaaattgagtggataaattatattacactttatatatacaataaatattttgttggttctatgtcaatgtactgattcatacaatttttaatatatgttaatttctttcattttcttttttaaggaaaaatcaatttgatttataaaaatcatcatatagcttctattatacagtttataattcatttacatattttgttgataacttttattgtaatttcttgtagcaaataaatatacgttaacactataattagaaagcaaaataaatcaaattaaatatataaaaataataataaatttttattttatttttttagccaatcaaaatcatgaaagcattttaaaaaattattaataaaactatattctttaatagaaataggatttgaattatataaaatatcaatataactaattaaattcaagatgtttaaaaataataatttttttatcttgttttttggctaaccaaattagaatgcaatttgtaattgaagaaatttaaccatactctttttaaattaaatatgaatcatctagatatatttatttaaaataaatttaaaaaaatgtaaaaatttttaaatgcctcatttacatatattatttaaaataataatgttgaagctaaattataaattattaaattattattaaaaatttacataataatataacgaaataaaatataattagacacaataagataataataataataatttaaaattttaaaatgcttaattatctatatttatttatctattaaaaaaagtaaaaaatccaatcaaatcataataatattatatatatatttaagttaaaactaaagttattatagaatatcttaaatttaacaaggataatatcggcaatcccattattcccctccttattcccacttatagatagacaagttatgatattatattttatataaaaactcaaattagttaaatttaaattgttatatggaataagtaattttattttcatagtttattatatattaaatttttgtctaattataatagtagtttatgttataaatgtgttatttccttaacaagttatttaattatttaattattattttattattttgtaaaataatttttttctattttgtgaatgctcttagattttagtattataaattgttataatatcatatttataaacaaaaactaatagaaaattatatatatacattaatagaaaaatttttatatagattaaattaaagtaaaaaaaagtacaaaactaaaaacataaaaataacacaaattaatattttgaagcattcatatatgtatgagagaatgttaaaaaaatttatgtgctataaaatagctaattgtttatgtaataaaatagcttttataatttagttacaattccttatattttttagtgtgctaattaaaaaatatatttattataataaatatggttaatattctgtgaaatagcttttactaattataatgtatatttttagtttgattgatgatgaagaaattcaataacagaataagattagataacttttattgtttaaatgtaattattttttcaacatttccttgtttaattaattatttaaattaaatattactaaaaataaattgaattcttatattcttatcatatttaaagtatatatatctaatatataaaaaaatattaattgagttattatatccttactatatttaacataccatatttaatatattatataactaattattttattagattaaattcttatatcttgccatatttaatatatagcttatttagtatataaattaattttttatgttatctaacataatttaaattcaaaattaatttaatttttttaatcaataaaattaaaatcttttaagatatattcttaacatatttaatatatatcttatttagtatacaatataatgttttattttatgtaatacaatttaaatttaaatagaaaattagtataaccaatcaaaattatattacctttgcttttactcaaccaatacaattaaaatttttagtcaatctataaacttagtatgaacgttttttagcaatctcaacgttctcttcccccatctgtacttttatacatataataaatagattataaataaggatagtaagtaagtgtatgaaagggataaaaaagggaatttactaagatagatatctatcggttaaatttgaaattcttgtcccaaatgcactttgtgcagagccattgtaatttctaattaaaaagagcaagatacaagaatggaccagtgattttaatggtgatgtagccatcaaagaataaagtttggatgggaggaaggagatgttgaactggtaaattgagggaaacgaaaatggtgaagatgataatctttgttgttttcattatcacaagttataatttacacttttgtcttttctattttaaatacactcatttatgtatctcaattgtaccatcttcctctcctttatatagacgttgcgtcgtattttttatctttataatattcttttatattgaaatgtcctgtttcattcatatgttataataaggattaagctgtttgtggataaaaataatataagtatttagtacttttatataaaactaatggaaatatatcgatgaaaaaaattagatacatagtcatgacggaactataccttaatgaccattataataatttaactatataggacaaaatcgttaatttaatcaaatctcatgaggaaatagtaacttttccaaaattttatgagccaagtactcgttatgcgtatcttaaaaaattcattttatctatctttataatattcttttatcttgaaatgttctgtttcatccataatttacagatgttataataaggattaatgtgtttgtggataagaataatataagtatttagtacttttatataaaactaatggaaatatatcgatgaaaaaattatatatatagtcacgacggaactataccttagtgaccattataataatttaactatataggacaaaatcgttaatttaatcaaatctcatgaggaaatagtaacttttccaaaattttatgagccaagtactcgttatgcgtatcttaaaaaaattcattttatgcgcataaaatataagtatatgagttatacacaaaaaatatttacacttttattttttaatattattattattattattattattgaaagcaaattattattattattgttattattatgaagcgttgaagaatgaatttaattggagtggaaaaaaaaaagaaaagaaaatgggagattctattagtggacaagggaagtaaaggtggatggtgagaacactatataaatacctaattgtacaacgcatctcgtatgataatacgagtgtagccaacaaaacaaccaataagatatataggtagtggtgcttccgtaatgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggtatttctttttattgttgttgttgttcttcttcttcttcttcttctttcagttgcttttgttttgtttttaaatctaaatgactatacaaacatgggttcttgggttttgaagtgggaattatgattcttttcttcttgaaattgcaggttatggtggaccccgacgcccctagcccaagtgaccctaatctcagagaatacttgcattggtaccttcatattcgatcccatatcatatgattctctcgacaaaattaatgcacacacaattttctttatttctcattcatatatgttcgtatccaatcatagttcaaacaaactttaaatccttcagtatacagatatatatggcttcgatccaatacttgtttcacaaaatcagtggaaaatgtgatttattagcaagttttctttatttatttaacatgccattccagttgaaggtaaattaaattaaatgataaggaagaaagaaagaaaacaaaaaatggaaatggcagagagcagtaggaaggaaaggtaaaaaaagctaagtacggaaaaagagaagtggtagtggcacaaaaacaaaaaaggtgtcgttgtagacaatgttatgctctttggggcgcggggtggtggggtttgaaagagacatatataggtgtcgtgtaatcacatcagtaagtcgttgctttttcaggttagagtaactgagtaagtatagaatatgtcaattttccctttgtctgttttgaaaattcatcgatgcatatttttctttcgagaaagagttcggaccgcacataatttgaagaaagtaggttcggatttactaggtttcttgggtaatcagttgggtcctacgtgtaccatgattctgcttctgggtacactatcttacatcacaacacaattcactaaattctgttctatttcaattttctactgtgggtcctgctttctacagtgattgattggagtaaggtgatccttgcgttgtctctttttacttttacttcattttaaaatggcaaccttttattattattattattattattataatataggtgcttgaggaaaaaaaaaaatttcttctttacaaaatattaaccgcacctatcattataaataaaaaaaatacttatttagatcagataactcaataaaaataaaaattttttaaaaaccattcatatttttttttctcagtaatatcctcttttaataaaaatttatttaattaatggatacattgtgcaccaggtttagataagtcaatctcttataagcaactcttttacgaacaataaatattttttgtaaaatatttgaaaatagtagcaaccacacctattattctgtaaaccagacttatttattttgtaaaccgaacaaatttaaaagataggcatattttacgaaataaattaaaaatattacaaaatataaattcttaaaaaccttacaaagtattagcgactgaaactgttatatattttgttaggcatgaagggttgggggaaccctcggttcaaaaattttaaattatggaattatgaaccggctttttaacggctgattctagttcggtttcaattttgactgattctgatgtggttttgatgaattgactattcctattatagttttgctatgtgtattatttttttttaaagaaaaataatttagttctaaggtgtttttctttcaaaaatattaatgtgaatttaaaaaaaatattgatagattttatctcaactaacttttaaagtgctcgtactcataagaaataaaattaattaattttgcaaaataatcaattaaatcatcattttatttttttataaatgttattaattaaataaattactagaaaattttgaaaaatgcttcaaataatgtaattttgaattaagtaaaaatttaaaaagagaaaatattaaactctcatttgtatcaaacattataattttatactactaaatatttctttttttttttgccattttactttttaatatttttaactttttaagtaaagtgtatttaaaaaaaaaaactaagtaagatgtattatgtagtaaaagattatttagaggtgtataaaataggttaaatgattataattttaaaaataaaaaaaatttaatttgaaccatattaaaactgccgatttcattcaggatcaaaatataccatttatatgaattttaatcgacttaaactatcaattttaatttaaaattttaaatagacagtttcaatcggtgaacagtttatttactataccgaactgatgcagatgtctatattttgtaatccgataactgcatgaaaaaatacaacaactttttactgacttgtcaagaaatgttagacgatagtaatgagtgctatcattttttaattacatgacatgacatgacatgacttttcttcacaccataatatgttggaccaagctagtcctaccactgtattacgttaggactcagtcagagagatggaataatggaacagaccaaaaaaacaaaacttttgttttaaattataatattcatattcattttgaggaaatcgaaatcgtttttggactcaaaagtgcaattccagtacgtatagtacgctgagaaagatgcaaaaggcgtctttgcagttgaagaacatttttaatatatatagggaagcatcaaccagtgcatgttcatgcagattttttttttttatttatttaaaaaaaaagaagatacaggcttaattatgatatgttgaatttatctaaacttgtgcaggttggtgactgatattccagcaactactggggtaacttttggtgagactttaattaattaattaattaattaagttttgtttaattatttagatgtcattttcttgaatctctccgttgagaccaaatcaagaatgtatatatgtgcagggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtgattcagttatatattaattactccaatctatacttgattaatcactaatgatcttaagaataagaagaagaagaagatgtattacggtaatgctatgcatggatccatgtgataatatttttatattttattgtggttctgatctctacatatatataattcaaccgaactaaaaagatcacaaatgctatcaaatttactgaatattaatctctattgcaatgtctttttcctttatttgaaatccaacgtaaagtatattttgaattagattcaacaatttctgataattatgtctaatgttatattttgaattagattcaacaatttctgataattatgtttaatgttatgttcactttttgaatttcatgtatgattcataaaatttgaacattgtcaacaaattaaaatctgaattaacaaatatatactttatgacacaacaatttccaaacttaaaattaaattgcccaagtacataactctcttactttaatcttatcgcgtcacccttaattttataagagacctatcatgcatctaaaattacgaattttgttaatgttgcacacatttttatgctaacactatttttttgagcttttgacttttatatgtaaaatgttgatttgatgtgtggttatcgtaatttttgaaataataattttcacattttttaatcatctaataagtatttaattggtttaattttataaatttaagtgttaaataaaataattttacaaattatagtatttaataagtggaattttatgagtcttaagcatttgatgtctcaaataaaaattgaaaatgcttcaataacaaagtttagagaggtttatgggcccaaattaaattatcaagtatgaatcacattcgggccaattaggttaattacatcaaagttgggcttgttttgttatggactggactagggaagattagaagttggactctgcgatggacgaaagctcaccaacctatagctgcagtagcctttacatgaaacgtgtcagattttgagagagagactcaccgtgactaaaatattaacgaacggaaacataaaaggcgccaaaactaaatatttggagccataacccaattgctgtagctggcgggaagacgttaaattattaattattccctccctttaacccaaaaaaaaaaaaagaaagaactaaaattaatatatttattctatcactctcatcactatcctatttgttttcttttttcttttttctgagtttctgaaaaactgaaaaatctaagatactaaaaaggtcaaaactctatgatgaatctgtgagacaggaacgaggaaatgaagtttcttgcttgaaaattcaaaccccctagatctcagtaacttgtgtcgtttctgtttctcttgctttctgcatcaaagatttttctgtttctgtctgaattagggttttgcttcttgcacaaataaagcaaatggcggaaccggtgccgacggtaatcgacgttgatagtgagccaaaacccaacacccaagaccagccaaagaaaaacctcaagcgaaagcgggcaacatcaacaccgtcgctgttatgcaacatgacggatgaccagaaggcagcccaaatcgagaccctgaaggacgaactccaggggctttttgtctattacagacaagagatggatcaagaactggggtttgggttcggtgcggatttgggtggaaatgaatgtaatacgctgaatgggatggtgggattgttgatggaggagagccaattggcgctgtccaaattggtggaggagatacatgcaaagttgtccaaggagagattgaaggacaatgtgactgttacggtggcggtggtgaagactgcggtgttgtttgtggggcagagaatgatgtacggagtgcccaatgtcgatgctgatgttttggaagatgagagtcaggattgtctctggtgttgggaggtattattagttttcatgggccctgtttaaattttattatattttagattgcattcttttatgtgaacaatgggtctatattggtaattgaattttatgaaattcacttaactattctgaattatgttaagttggatatcatgtttctatcattccggggaatttaatttattaagccttttatgttgtgttcagtttttatttatattgatttcttcgttgaggaatttcttttccttggtagtggagcgttcagtgggaatttcttgtgttgctaggttattggttataattgctgtttttgattttgagtgtgagcaatctaacctttgtaggcgaactgtgaacaaaagaagctaggcgggattttggcacttactattacaagaaagctgccctgtaaattctgaagtcttgggttcaccagagtacttgttcactgtgttttctagagtgtgcatttattatgtgtcaataatttattattgttctgcaaatcatcctgttgaatgtgtgtgcttggtttgctatcaggacatctttatattctaataccaatttctgtccttcggcggtttcctctgctgttggttgtatttcatcttgataaccagaaatgtgattttcatacttactgttgcagacaagagatctaaaggtgatgccaaaatacttgcgtggaactttaaaagttcggcggatctgtcggaaaaagattcatgagagaatcagtgctgtgtctggtagaaaacattgccttgaatttatcaattcttttatttgttcacttgttgttttgagatttttcattttcttattgtgactgtaatttatgaacaatttaatgtggtttgatgttactaaagtgaagattaaggtagtctagccttttagtctaagaggagaaatatatgaatcattttactgcaccagtaaaacaaatgaaaatgaatctttgaatttagataatgtttgggttatttcagctatgatatctgcattacaaaactcggagacttatcagagttgcagaactgatttaatgagagcatcaggaaagcttgctaaagctttaaaagaggcagaaattcgttcattagtggatggaacgttacagaagaatggtacagtgaagtatgtctctgatttgctctattctgtttatttcctaaatatgtttttttttctttattaaactatttgagtatcacagggctgatcaggaagcaaaactagaacagaaagtgctcatcaagcaattggagaaaaataaacgagaagttgaaaaggagaaaaagaggatggatcttgagcttcaaaaggaaaagaggcaaattgtatgtgtgaatctcacagtttttttagtatatgcattcattctctagtgtcaacaagatgacactttgttcatataatataattagaaatatgtaaaaagtaaagcctttttttcgttcagaatgctaaatgataactatgacttgggcagataaaaaaacccataagtgtgtgtatgcatcatatgtaaaaaactatatatatcatgtttacactgagcaaaggatttccattttgctgtattttcagaatgttttccatgtcatagatttttttttttttttttcaggaaaaagagcagaagcgattgcaggaggaggcagaaaaggatgaaaagcgccgcgaaaaagaagagtctgaaatgaggagacaactaaaaaagcagcaaaaggaagttgagaaagaacaacggcataaagagaaggaagaagctaaaatgaaaaggcaaaatgctataaagaagcaagcttcaataatggaacgctttcttaaaagaagcaaaactgattccccatgccaaaatgagggaacttcaattgaagaaaccgcacctgttttgtcaggcaagaagagtgagaagatgcctgaggcagttaccatggcaatggattgtactctgtcatcaaacgatgatattagaattgatgatattcgcaagtagcttccttactccctaaaactttttttttttcttttcatttcatttttgaccctttttgggtattcaacgattggatttgatgattaagattgatgggctatgcaggctgcacttatcttcttggcatcacctaggtcatgctattcgttcaaataggaagcagcattggagcattcgtcaaaagcctaagactgaattatttaaggaactcaagctaactactgccagagaactttctcacgatggtgagttgattgtagagaagcttgaaagtgaatggggggaacagtcttctgatgatagattatgtgcaacaaatttagagagttctctgaatgataaaaaatggaaaaggagaaagaagctgttgcagtttgataagagtcataggcctgcattttatggtatttggccgaagaaaaggtgaactcttaactgtgattttattatgtttcacaaaaaccacaaactaaattagatgagctatggaaatgtacaagagtcatatgctttaaaactgaatctctgtaaaattatggaaatagaaatactcttttttttctgtctagtctgcttatcatgttaagttgtactacaaggtaatgccatgtgccttgattaattggttcacacacagagacatatatgcatattctgaagttaatcctgtttagatttattcaaagtgcaagataggaatgcttacagttttgagggccaactgattttatattctgaaagaaaatcttgtgctaggaagggtggatgtccaaatgcacaaacatgtttatatttttggaggctctaggctcaaaaacaaatcttgtttcatttttagccaaatttttttattgtttttacattttgtttgatgacttgataaggcatctattcaatagttgttcatgagtgatactcgatttgtcatctatggtagttggtaaatctatgggtgtttttaaactgctgaaagatctgacattcgatctaattcactgttttgctgtgtctttaagtttcttgtgtgcatttaatggatatatatatatatatatatatatatatatatatatatatatatatatatatatat

SEQ ID NO: 4 - DNA, 13848 bp, Artificial Sequence
  pX7 vector with Jatropha curcas JcFT coding sequence
ctagtcgatccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcgagggggtaccgagtcgaggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgggtaccgagctcgaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactatagtttaaactgaaggcgggaaacgacaatctgatccaagctcaagctaagcttgcatgcctgcaggatatcgtggatccaagcttgccacgtgccgccacgtgccgccacgtgccgccacgtgcctctagaggatccatctccactgacgtaagggatgacgcacaatcccactatccttcgcaagacccttcctctatataaggaagttcatttcatttggagaggacacgctgggatcccctaataacttcgtatagcatacattatacgaagttatgaattaaatccgggcggaatgaaagcgttaacggccaggcaacaagaggtgtttgatctcatccgtgatcacatcagccagacaggtatgccgccgacgcgtgcggaaatcgcgcagcgtttggggttccgttccccaaacgcggctgaagaacatctgaaggcgctggcacgcaaaggcgttattgaaattgtttccggcgcatcacgcgggattcgtctgttgcaggaagaggaagaagggttgccgctggtaggtcgtgtggctgccggtgaaccgtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggcgaattcccagagatgttagctgaaatcatcactaatcagataccaaaatattcaaatggaaatatcaaaaagcttctgtttcatcaaaaatgactcgacctaactgagtaagctagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaacaagcttggaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatccgctagaggatctcgacctgcaagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgggatcttggaggtgatgtaacatgatcacaagctgatcccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattgatcccccctcgacagcttgcatgccagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgctcgactctaggatcttcgcaagacccttcctctatataaggaagttcatttcatttggagaggacacgctgaagctagtcgactctagcctcgacatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggtaagtcttcttttcctttactctttacagaaatggtaatctcagatatagtaatggataagatccaaaaatgacacttttaaccaagattgtacgaagatctttttaaactccattttttattttgacatctaaattggatttaactcggccttgctgtattttggcaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagtaagaattcgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcataacttcgtatagcatacattatacgaagttatagatcttcgacctcgagatgcctagggatcaatttagagaccctctcgttgttggtcgtgtgattggggatgttttagacccttttacaaaatctatctccctccaggttacttataatcacagagaggttaacaatggctgtgagctcaaaccctctcaagttgtcaaccaacctagggttgatatcggtggagatgatctgaggaccttttatactttggttatggtggaccccgacgcccctagcccaagtgaccctaatctcagagaatacttgcattggttggtgactgatattccagcaactactggggtaacttttgggcaagagatagtgtgctatgagagcccgcgaccatcgttggggattcatcggttcgtatttatattgttccggcagctgggaaggcagaccgtgtatccaccagggtggcgtcagaatttcaacactagagatttcgctgagctctacaatcttggttcaccggtggctgctgtttattttaattgccagagggagagtggcaccggaggacggagacggtga

SEQ ID NO: 5 - protein, 173 aa, Arabidopsis thaliana
MAASVDPLVVGRVIGDVLDMFIPTANMSVYFGPKHITNGCEIKPSTAVNPPKVNISGHSDELYTLVMTDPDAPSPSEPNMREWVHWIVVDIPGGTNPSRGKEILPYMEPRPPVGIHRYILVLFRQNSPVGLMVQQPPSRANFSTRMFAGHFDLGLPVATVYFNAQKEPASRRR

SEQ ID NO: 6 - protein, 172 aa, Jatropha curcas
MAASVDPLVAGRVIGDVIDLFVPSVTMSVYYGSKHVTNGCDVKPSTASNPPKLTISGHPNDLYTLVMTDPDAPSPSEPSMREWVHWVVADIPGGTNPTKGKEILSYAGPRPPVGIHRYILVLFRQKAAMGVVEQPQSRANFNTRLFAAHLELGLPVATVYFNAQKEPAAKRF

SEQ ID NO: 7 - protein, 174 aa, Picea abies
MARSTDPLVVGRVIGDVIDMFVPSNDMAVYYGSKQVRDGCEIKPSATVDRPKVQIAGRHFDDSLYTLVMTDPDSPSPSEPNMREWVHWVVTDIPGATDAAQGREILPYMGPRPPIGIHRYIFVLFKQSGPMVMMVPPQARNNFSTRAFASEYSLGLPVSAAYFNAQKEPGTRKR

SEQ ID NO: 8 - protein, 175 aa, Jatropha curcas
MARSLEPLVVGKVIGEVLDMYNPVAEFTVHYGSKQVANGCEIKPSVAAQKPHVHILGSRLSSDLYTLVMVDPDAPSPSEPKWREWLLWIVVDIPEGSDATKGHELVSYMGPQPPTGIHRYVFALFKQKGALMGRIQPPDGRGNFNTRHFAAQSGLGLPVAAVYFNSQKEPAVKKR

SEQ ID NO: 9 - protein, 175 aa, Manihot esculenta
MARSLEPLVVGKVIGEVLDMYNPVAEFTVHYGSKQIANGCEIKPSAAAQKPHVHILGSRLSSDLYTLVMVDPDAPSPSEPKWREWLHWIVVDIPEGSDATKGHELVPYMGPQPPTGIHRYVFALFKQKGALKGRSLGPDGRGNFSTRQFAAQHGFGVPVAAVYFNSQKEPAVKKR

SEQ ID NO: 10 - protein, 175 aa, Ricinus communis
MARSLEPLVVGKVIGEVLDMYNPVAEFTVHYGSKQIANGCEIKPSAAVQMPRAQILGSRLTPSLYTLVMVDPDAPSPSDPKWREWLHWIAVDIPEGSDATKGHQLVPYMGPQPPTGIHRYVFAVFKQGEALNGRTKAPEGRGNFSTRQFAAQNGLGLPVAAVYFNSQKEPAVKKR

SEQ ID NO: 11 - protein, 172 aa, Picea abies
MSRFVEPLVVGRVIGDVLDMFVPSVDLAVTYASRQVNNGCELKPSAITLLPRVDIGGEDLRNFYTLVMTDPDAPSPSDPTLREYLQWIVTDIPATTSASFGRELVSYESPRPTIGIHRFIFVLFKQMGRQTVYPPGSRLNFNTRNFALSNSLGLPVAAVYFNAQKEAAGRRR

SEQ ID NO: 12 - protein, 175 aa, Arabidopsis thaliana
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQLGRQTVYAPGWRQNFNTREFAEIYNLGLPVAAVFYNCQRESGCGGRRL

SEQ ID NO: 13 - protein, 176 aa, Jatropha curcas; identical to SEQ ID NO: 2

SEQ ID NO: 14 - protein, 175 aa, Manihot esculenta
MPRDRDPLAVGRVIGDVLDPFTRSISLNITYNNRDHVTNGCELKPSQVVNQPRVDVGGDDLRTFYTLVMVDPDAPSPSDPNLREYLHWLVTDIPGTTGASFGQEVVCYESPRPSVGIHRFVFILFRQLGRQTVYAPGWRQNFNTRDFAELYNLGLPVAAVYFNCQRESGSGGRRR

SEQ ID NO: 15 - protein, 177 aa, Arabidopsis thaliana
MENMGTRVIEPLIMGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 16 - protein, 177 aa, Euphorbia esula
MSNNNNKHNDPLVVGRVIGEVIDSFNPSVKMTVTYNSNKQVFNGHELFPSAVTFKPKVEVQGADMISFFTLVMTDPDVPNPSDPYLKEHLHWIVTDIPGTTDATFGKEVVSYEMPKPNIGIHRFVFLLFKQKRRQTVKTPTSRDKFNSRKFAVDNELSLPVAAVFFNAQRETAARRR

SEQ ID NO: 17 - protein, 175 aa, Jatropha curcas
MAKVSDPLVVGRVIGDVIDYFTPCMKMTVSYNSNKQVYNGHELFPSAVTHKPKVEVQGADMRSFFTLVVMTDPDVPGPSDPYQREHLHWIVTDIPGTTDATFGGKEVVSYEMPRPNIGIHRFVFILFKQKRRQMVNTPTSRDKFNTRKFAEENELGLPVAAVFFNAQRETAARRR

SEQ ID NO: 18 - protein, 173 aa, Jatropha curcas
MEKPVDPLIVERVIGDVLDLFTPTIKMSVAYTDRKVCNGHELYPSTIASKPKVAVEGDDMRSFFTLVMTDPDVPGPSDPYLREHLHWIVSDIPGTTDATFGKEIVSYEIPRPNIGIHRFVFVLFKQKKRSQIQHPPWSRDNFNTRNFAAENELGLPVAAVYFNAQRETAARRR

SEQ ID NO: 19 - protein, 173 aa, Ricinus communis
MSRAVESLVVGRVIGDVVDSFTPMLNMSISYGNRRVFNGYELHPSLVALKPKVEVQGGDMRTFFTLVMTDPDVPGPSDPYLREHLHWLVTDIPGTTDATFGREVVTYEIPRPTIGIHRFVFVLFQQKRRQTINPPSSRDNFSTRDFAVGNDLGLPVAAIYFNAQRETAARSRR

SEQ ID NO: 20 - DNA, 30 bp, Jatropha curcas
ataagtcgacatgagggatcaatttagaga

SEQ ID NO: 21 - DNA, 32 bp, Jatropha curcas
ttatttctagatcaccgtctccgtcctccggt

SEQ ID NO: 22 - DNA, 30 bp, Jatropha curcas
ataactcgagatgagggatcaatttagaga

SEQ ID NO: 23 - DNA, 32 bp, Jatropha curcas
ttattactagttcaccgtctccgtcctccggt

SEQ ID NO: 24 - DNA, 27 bp, Jatropha curcas
aaagtcgacatggaaaaaccagtagac

SEQ ID NO: 25 - DNA, 27 bp, Jatropha curcas
aaatctagagcgtcttcttgcagcagt

SEQ ID NO: 26 - DNA, 27 bp, Jatropha curcas
gaagtcgacatggcaaaagtgtcagat

SEQ ID NO: 27 - DNA, 27 bp, Jatropha curcas
aaatctagagcgtcttcttgcagcagt

SEQ ID NO: 28 - DNA, 27 bp, Jatropha curcas
aaagtcgacatggcggcctctgttgat

SEQ ID NO: 29 - DNA, 26 bp, Jatropha curcas
gaatctagaaaaccttttggctgctg

SEQ ID NO: 30 - DNA, 24 bp, Jatropha curcas
aaagtcgacatggctcgctctctt

SEQ ID NO: 31 - DNA, 25 bp, Jatropha curcas
gggtctagaaacgttttttaactgc

SEQ ID NO: 32 - DNA, 528 bp, Jatropha curcas
  CDS (1)..(525)
atggcaaaagtgtcagatcctcttgtggttggaagagttattggagatgtgattgattatttcacaccatgtatgaaaatgactgtttcttataactccaacaagcaagtttataatggtcatgagcttttcccttctgctgtaactcataaacctaaagtggaagtgcaaggagctgatatgagatcctttttcactctggtagtcatgacagacccagatgtgcctggaccaagtgatccataccagagggagcacttgcactggatagtgacagacatcccaggaacaacagatgccacatttggtggcaaagaagtggtgagctatgaaatgccaaggccaaatatagggatccataggtttgtgtttattcttttcaagcagaaaagaaggcaaatggtgaacacaccaacatcaagagataaatttaatacaagaaaatttgcagaagaaaatgaacttggcctgcctgttgctgctgtcttctttaatgctcagagagaaactgctgcaagaagacgctaa

SEQ ID NO: 33 - protein, 175 aa, Jatropha curcas; identical to SEQ ID NO: 17

SEQ ID NO: 34 - DNA, 2024 bp, Jatropha curcas
ttcaaaattatgtgttataaatcaaaaaactgcaaaattggtcaacaaaaagaaggaaaagggaaggatggaaagaaaaagagagagagagtacttgttaaataagttaatttgtcatcaaaaattgaaatactaataaaaaaaaggttgatttttgagtttttaaaatcagactcaaatttagggagcatattaccattatttataattttaatgtgcgtccttttattaatattaaataaaagattgtccaaactcgatttgtaggacaatccataaactaaaccaatccaaatagtaatatttgagcaagtcaatcccataaaaattgtgtacaaaatccttcctatttcatctcctatcaaccaaaacaaacataaaaatacaaataaattgtctttttttttttaagaatttaaataaattgtcttatgatacccatttacctcagtcattatactcatcagactcaatatattaaattcacagtgaattttctaaaaatttatattcattatataaattgtaattcaaatagcttactattcatcaaggactaagaaagtccaataggtcccagccaaagaacattcccacagtagacaaccaaaacaccaatgatgtatttattgactcttgtaagaaacccctccaatcctaatatacagtgggttcagtagctagggctaatcaacactataaataagggggttccactctctttgccttgcaagaaatatcctatagacattttccacagcaaacaagaaaataataattagcttattccagtcccaaaattacattgtcatccttatctctttctttttctttctctgtctctctctctctctctctctcttatatctctaccaactaagttacagtgtttcttgggtagtctttatatggcaaaagtgtcagatcctcttgtggttggaagagttattggagatgtgattgattatttcacaccatgtatgaaaatgactgtttcttataactccaacaagcaagtttataatggtcatgagcttttcccttctgctgtaactcataaacctaaagtggaagtgcaaggagctgatatgagatcctttttcactctggtaactaaatcaaactcatattaatttattattctatatagttctcctcttgatattatctctgtctgtgtgtgtgtgtatatatatatataatggatttatcttaccactttcatttcaggtcatgacagacccagatgtgcctggaccaagtgatccataccagagggagcacttgcactggtattattactagtgtgtgttttttcttttttaatctacacataaacacacacccacaaattatctagtgaattttttttctctctctctctctaaatgaaaatgaaagatcatggaaaaagaagaggaaaaagaagaaaatgagtttattgatatatattgcaagtacaattatggcaaaaatagaaagcataattgattttagcatttccaaattttcataaaacttaagaaagtagagaaacaaacatagtagggaaagtacatattaatatatgcatatttagcaacttatattctctgaaggactgaacttgatttgaccattttacaggatagtgacagacatcccaggaacaacagatgccacatttggtaagcttcactacacatatatggatatacaatacacacaaacagagagagaagataaataattattttatgggttttatttgaaatgatagattatttataattaaaaaaaattgcaggcaaagaagtggtgagctatgaaatgccaaggccaaatatagggatccataggtttgtgtttattcttttcaagcagaaaagaaggcaaatggtgaacacaccaacatcaagagataaatttaatacaagaaaatttgcagaagaaaatgaacttggcctgcctgttgctgctgtcttctttaatgctcagagagaaactgctgcaagaagacgctaaggggaaggaaaagtacacaa

SEQ ID NO: 35 - DNA, 522 bp, Jatropha curcas
  CDS (1)..(519)
atggaaaaaccagtagaccctcttattgttgagagagtgataggagatgttcttgatctttttacaccaactataaaaatgtctgttgcttatactgacaggaaagtctgcaatggacatgagctttatccatcaactattgcttcaaaacctaaagttgctgtggaaggagatgatatgagatctttctttacattggtgatgacagacccagatgttcctggtcctagtgatccttatttgagagagcacctgcactggatagtgagtgacattccaggaacaacagatgccacatttggaaaggagatagtgagctatgaaattccaaggccaaacataggaatccacaggtttgtgtttgtgttgttcaaacaaaaaaaaagatcacaaatacagcacccaccttggtcaagggataatttcaacactcgaaattttgctgctgaaaacgagcttggtcttcctgttgctgctgtttacttcaatgcacaaagggaaactgctgcaagaagacgctaa

SEQ ID NO: 36 - protein, 173 aa, Jatropha curcas; identical to SEQ ID NO: 18

SEQ ID NO: 37 - DNA, 4275 bp, Jatropha curcas
ttcaaagtttgcttaaaaaaagagaataaaatgttgaaaaattagacagccaagcgaattattctcattatttcactaaaaatatattttttaaaaaatctcatcattccccttatttacacatctttatatatagatattatagatgaagctcatcatcatcaagaacaatgaagagaaatttctttgaagctttaatcttttcctgttcttgcatttttttttttcttttagtagaaagttagaaacagaaatggcaacttgtggtttttttagtaaggatccaaatcctgtagggttctacaaatactaattcacttctcaatctagcaaatatgtaattacaagtaataagttgattttgcatgtacatgccaatagctatttggaatggtgtaaggtagcgcagtttaatatgttaggattggaattcacttaatactaaaattgaaatcatttttgtataattagtacattttaaatgttggtgaaaaaaataacttgggactattataattgggaatttgcggctacatgacttttcatgtagcgctacggtactttgattaaggtcaaaacactacgtattatttatagtgttcaataacataatgtttttattaatatttaaagtttaataaaatagattataaaatgttactttctttgcctatgtattattatattaaataatttaaaaattctctctataagatattttttatacaaaaattatttcaaattttattttcaatttatactctaatttgatttgttattaatttgatcaaaacaaacattggagttaaagaattcatttaaccgaccctaaataatttggaactaagttttagttgttgctgttgtctgccttgacaataattataattcttgatagtcaatgaattaatagcatgtatttatatatttttaatttttttaattattatcattatcttaaatttcctaaggtttgtaaaaaaaaaaattacatataacttttttaaaaatttccaacatttatagagaagctaagtttacttcccactaaaaataggcattaactctcatgctgggttttcatcagttgcacttttcttttccaagtctctccctttggatgaatcatcatttaagacaagtggtagcctattaatttgtttcttctttttaaacttcaattttctttctgttatataaaaagaaaaagattactgttctcgaaaataacctttttttttaaaaaaaaagtttccattgatatattttcttaaatacagcaacactagaaatacaaacatatcatgaattgaagatttgatcgcttgctggagttgaatctttttcactcagactaatctatgttcataaatattaatgtttcttaatcaatccagttcaaaaatatcatctcaacggcaattaagtccttatttaatctaggattcaaaccctaagcaagctagcttgcagactgacctatcacttggatgatttatttttttctttccaaggaacactgatgagtagtaactagctaggaagagacagcccccaaactaagaaccaattcctagggacccaagaaaacaagcaagggaagagatgttaattaggggtcacaaaaacatataaacacagctgataagatgcaaatggaaaatataattatttaaatttgtggtttacaatacaaagtctagcccaggaatttatacctattgaatgagactgagaaacgaatcagttaagtacgatatatattcacatattccataatattattaatttgttctagtttttctaccactgctgactcctgtaaattactctcaacttacagggcactccctataaatattaggccaggcctctgctcaactccattataacagaaaaaggaaacatagccttactaattaagcttctctctctctctccctagctatctccattatctatcaatggaaaaaccagtagaccctcttattgttgagagagtgataggagatgttcttgatctttttacaccaactataaaaatgtctgttgcttatactgacaggaaagtctgcaatggacatgagctttatccatcaactattgcttcaaaacctaaagttgctgtggaaggagatgatatgagatctttctttacattggtatgtatttaggcctcttataagattgaaacttgtttctctctcttttttttttttttttttttgttttattttttattttttataatttgtgagactatatataattttattcttttcaatcaggtgatgacagacccagatgttcctggtcctagtgatccttatttgagagagcacctgcactggtatacttagaccactaatttttctacttcaattgtgtttatattttaggtatatcctagctatttagggttcattaaccttcagttctagggcaaaacttcttacatagagtttatacttacctttgattaaatgatgctgaagtaataaataaatcctacaacaaattcatatgtccatagcaatgaggacgaatactggtgcttgtaagtttcgagctttttacttgaaatctttaattttatattaaacttgaaattttttttttttttgcattagtgagaaactatgtctatctgtcttattttcttttttctgattaactgcaaaaattttattaaaaaatgaggaaaaatcatctgcaatttagctgtttataaaaaaaaaaagaaaaagagagaaagaaagaaaggaagaaaatgctgaaattgattcagactttaactgagtgaatcaacagattttacaatctagaagggaaaaaaaaaaaaaaaatcacccggtacatattaacaatagctacaagataaagaaatcatcaacaccacaagatgaacaaatctagctacatatatacatatatagaaagagagatgaaaattaattttatggcattgcaagaaatagcaaaatatataaaacaaatagatacgttgttgtcagattatctaaagaaattactttaattgaacttttttgcaggatagtgagtgacattccaggaacaacagatgccacatttggtgaattcatactcgttaattcaattttacttccttccaaaagaaaatgctaattaaaatgtttacgtacaacaggaaaggagatagtgagctatgaaattccaaggccaaacataggaatccacaggtttgtgtttgtgttgttcaaacaaaaaaaaagatcacaaatacagcacccaccttggtcaagggataatttcaacactcgaaattttgctgctgaaaacgagcttggtcttcctgttgctgctgtttacttcaatgcacaaagggaaactgctgcaagaagacgctaattttaacaattaatttcaagattatatctcttgaaattataataatttctatctctaaaatatgaatgtatcgtaacttattatctttgtttcggatgttttgaagatttgtgggttgcagtctctttcaatatatatgtttgttacagaagatgagcagtaaaatattataaatgctaataacttatttagttctctgtttgtaatttttccttaactctatttttttttttttaagctaactttatatgtaaaatcaagagtttaaaatttaaattcatcactgtggagacaattttttaattaaatgaatattactctcaatccaaacctaaaaatgtacatgtgtaaataaaaaaaaaaaaggagaaaaaaaattcagatcttatttatattatcatcatattttatctgtagctacctatcaactttgaacttacaaatatgttttaatgcaaaatatttttcatattttatgaatataaaggaagatttatcttttaagctcactatcaaaaaaagaacattaattagtgacagaaaaatctttcgcaatttaaatgttaattagcgacagacaaaaatctttcgcaattttctgataggacttttggcgattgattttatatttcagaaaaagaaattttttagtgaaatttacgatgggattgcgatgactaattcaactatactagaaagggttaattataaatttagtatccgaacttctcaattttttttttttttttttttggtctcataagctggagaactttattaatggtgaagagaaaaacaacttgagcttaaaaactacacctgagccaaatggactcatccaaaacatgcaaagcctgacttgggaacaagaaacccatagagvgg